(19) United States
(12) Patent Application Publication
(10) Pub. No.: US 2010/0254983 A1
(43) Pub. Date: Oct. 7, 2010

(54) USES OF RAGE ANTAGONISTS FOR TREATING OBESITY AND RELATED DISEASES

(76) Inventors: Ann Marie Schmidt, Franklin Lakes, NJ (US); Anthony Ferrante, Montclair, NJ (US); Fei Song, New York, NY (US)

Correspondence Address: COOPER & DUNHAM, LLP, 30 Rockefeller Plaza, 20th Floor, NEW YORK, NY, 10112, US

(21) Appl. No.: 12/451,894

(22) PCT Filed: Jun. 6, 2008
(86) PCT No.: PCT/US2008/007143

§ 371 Date: Apr. 22, 2010

Related U.S. Application Data
(60) Provisional application No. 60/933,754, filed on Jun. 7, 2007.

(51) Int. Cl. A61K 39/395; A61K 38/16; A61K 31/415; A61K 31/695; A61P 3/06; A61P 3/04; A61P 3/10

(52) U.S. Cl. 424/134.1; 514/4.8; 514/6.8; 514/399; 514/63; 514/7.4

(57) ABSTRACT

This invention provides a method for treating obesity in which comprises administering to the subject an antagonist of a receptor for advanced glycation end products (RAGE) in an amount effective to inhibit binding of a ligand of RAGE to RAGE so as to thereby treat obesity in the subject. The present invention also provides a method for treating hyperglycemia and increased cholesterol, insulin, triglyceride and leptin levels comprising administering to the subject an antagonist of RAGE in an amount effective to inhibit binding of a ligand of RAGE to RAGE so as to thereby treat hyperglycemia and lower cholesterol, insulin, triglyceride and leptin levels on the subject.

This invention was made with support under United States Government Grant No. HL60901 from the National Institutes of Health. Accordingly, the U.S. Government has certain rights in this invention.

Throughout this application, various publications are referenced by author and date. Full citations for these publications may be found listed alphabetically at the end of the specification immediately following the Experimental Procedures section and preceding the claims section. The disclosures of these publications in their entireties are hereby incorporated by reference into this application in order to more fully describe the state of the art as known to those skilled therein as of the date of the invention described and claimed herein.

### BACKGROUND OF INVENTION

The Receptor for Advanced Glycation Endproducts (RAGE), a multiligand member of the immunoglobulin superfamily of cell surface molecules (Schmidt et al., 1992; Neeper et al., 1992), interacts with distinct ligands implicated in development and homeostasis (Hori et al., 1995), as well as in certain pathophysiologic situations, such as diabetes, Alzheimer's disease and inflammation (Park et al., 1998; Wautier et al., 1996; Yan et al., 1996; Yan et al., 1997 and Hofmann et al., 1998).

The extracellular (N-terminal) domain of RAGE includes three immunoglobulin-type regions: one V (variable) type domain followed by two C-type (constant) domains (Neeper et al., 1992; Schmidt et al., 1997). A single transmembrane spanning domain and a short, highly charged cytosolic tail follow the extracellular domain. The N-terminal, extracellular domain can be isolated by proteolysis of RAGE or by molecular biological approaches to generate soluble RAGE (sRAGE) comprised of the V and C domains.

RAGE was first identified as a signal transduction receptor for products of nonenzymatic glycation and oxidation of proteins/lipids, the Advanced Glycation Endproducts, or AGES, whose accumulation in disorders such as diabetes has been linked to the pathogenesis of vascular and inflammatory cell complications (Brownlee et al., 1988; and Sell and Monnier, 1989). RAGE is expressed on multiple cell types including leukocytes, neurons, microglial cells and vascular endothelium (e.g., Hori et al., 1995). Increased levels of RAGE are also found in aging tissues (Schleicher et al., 1997), and the diabetic retina, vasculature and kidney (Schmidt et al., 1995). Subsequent studies identified RAGE as a neuronal/microglial interaction site for amyloid-beta (A(3) peptide (Yan et al., 1996; Yan et al., 1997), the proteolytic cleavage product of beta-amyloid precursor protein, whose accumulation in Alzheimer disease brain has been linked to inflammation and neurotoxicity (Selkoe, 1994; Sisodia and Price, 1995). More recently, Extracellular Novel RAGE binding protein (EN-RAGE)(Hoffman, et al., 1998), members of the S100/calgranulin family of proinflammatory cytokines (Schafer and Heinzmann, 1996; and Zimmer et al. 1995) and High-Mobility Group Box Chromosomal protein 1 (HMGB1), a protein with both intranuclear functions and extracellular cytokine-like effects (Hori, et al., 1995; Kokkola et al., 2005), have been identified as ligands for RAGE. Interaction of these ligands with RAGE triggers proinflammatory pathways in endothelial cells, macrophages and lymphocytes while blockade of RAGE suppressed the immune/inflammatory response in murine models of delayed-type hypersensitivity (DTH) and colitis (Hofmann, et al., 1998).

Despite the broad expression of RAGE and its apparent pleiotropic role in multiple diverse disease models, RAGE does not appear to be essential to normal development. For example, RAGE knockout mice are without an overt abnormal phenotype, suggesting that while RAGE can play a role in disease pathology when stimulated chronically, inhibition of RAGE does not appear to contribute to any unwanted acute phenotype (Liliensiek et al., 2004).

### SUMMARY OF INVENTION

This invention provides a method for treating obesity in which comprises administering to the subject an antagonist of a receptor for advanced glycation end products (RAGE) in an amount effective to inhibit binding of a ligand of RAGE to RAGE so as to thereby treat obesity in the subject. The present invention also provides for the antagonist to be a fusion peptide of RAGE or a small molecule.

The present invention also provides a method for treating including hyperglycemia and increased cholesterol, insulin, triglyceride and leptin levels comprising administering to the subject an antagonist of RAGE in an amount effective to inhibit binding of a ligand of RAGE to RAGE so as to thereby treat hyperglycemia and lower cholesterol, insulin, triglyceride and leptin levels on the subject.

### BRIEF DESCRIPTION OF THE FIGURES

FIG. 1. Treatment of wildtype C57BL/6 mice on a high-fat diet with sRAGE prevents weight gain over time. Wild-type C57BL/6 mice were started on a high fat diet on day 1 of the experiment. On day 31, the animals were treated with either soluble RAGE, 150 μg every other day by intraperitoneal route, or by vehicle, phosphate buffered saline (PBS) (equal volumes per day). The weights of the animals were recorded over the course of the experiment. The gray diamonds correspond to the PBS treated animals and the black squares correspond to the sRAGE treated animals.

FIG. 2. Treatment of wildtype C57BL/6 mice on a high-fat diet with sRAGE reduces the weight of epididymal adipose tissue. Tissue weight is represented comparatively with PBS-treated animals on the left and sRAGE-treated animals on the right.

FIG. 3. The ratio of epididymal adipose tissue weight to total body weight is lower in sRAGE treated wildtype C57BL/6 mice on a high fat diet as compared to vehicle (PBS)-treated mice. The ratio of adipose tissue weight to total body weight is represented comparatively with PBS-treated animals on the left and sRAGE-treated animals on the right.

FIG. 4. RAGE null mice on a high-fat diet fail to develop hyperglycemia. Wild-type C57BL/6 mice were fed either regular chow (“B6/Reg”, X's) or high-fat diet (“B6/Fat”, triangles). RAGE null mice were fed either regular chow (“RKO/Reg”, squares) or high-fat diet (“RKO/Fat”, diamonds).

FIG. 5. RAGE null mice on a high-fat diet fail to develop obesity. Wild-type C57BL/6 mice were fed either regular chow (“B6/Reg”, X's) or high-fat diet (“B6/Fat”, triangles). RAGE null mice were fed either regular chow (“RKO/Reg”, squares) or high-fat diet (“RKO/Fat”, diamonds).

### DETAILED DESCRIPTION OF THE INVENTION

This invention provides a method for treating obesity in a subject which comprises administering to the subject an antagonist of a receptor for advanced glycation end products (RAGE) in an amount effective to inhibit binding of a ligand of RAGE to RAGE so as to thereby treat obesity in the subject. In one embodiment, the RAGE is human RAGE. In one embodiment, the antagonist is a polypeptide. In one embodiment, the polypeptide is a soluble fragment of RAGE. In one embodiment, soluble fragment of RAGE is sRAGE. In one embodiment, the soluble fragment of sRAGE is a V-domain of sRAGE or a fragment of the V-domain which retains the ability to inhibit the binding of a ligand of RAGE to sRAGE. In one embodiment, the V-domain of RAGE comprises consecutive amino acids comprising the sequence A-Q-N-I-T-A-R-I-G-E-P-L-V-L-K-C-K-G-A-P-K-K-P-P-Q-R-L-E-W-K (SEQ ID NO. 6). In one embodiment, the fragment of sRAGE is a fragment of the V-domain of RAGE which comprises consecutive amino acids having the sequence A-Q-N-I-T-A-R-I-G-E (SEQ ID NO. 7).

In another embodiment, the antagonist comprises a fusion protein comprised of a RAGE polypeptide linked to a second, non-RAGE polypeptide wherein the RAGE polypeptide comprises a RAGE ligand binding site. In one embodiment, the RAGE polypeptide is linked to a polypeptide comprising an immunoglobulin domain or a portion of an immunoglobulin domain. In one embodiment, the polypeptide comprising the immunoglobulin domain comprises at least a portion of at least one of the C_{H}2 or C_{H}3 domains of a human IgG. In one embodiment, the RAGE ligand binding site comprises consecutive amino acids comprising the sequence A-Q-N-I-T-A-R-I-G-E-P-L-I-L-K-C-K-G-A-P-K-K-P-P-Q-R-L-E-W-K (SEQ ID NO. 6) or a sequence 90% identical thereto or Q-N-I-T-A-R-I-G-E-P-L-V-L-K-C-K-G-A-P-K-K-P-P-Q-R-L-E-W-K (SEQ ID NO. 8) or a sequence 90% identical thereto.

In one embodiment, the RAGE polypeptide comprises consecutive amino acids corresponding to amino acids 24-116 of human RAGE (SEQ ID NO: 9). In one embodiment, the RAGE polypeptide comprises consecutive amino acids corresponding to amino acids 24-123 of human RAGE (SEQ ID NO: 10). In one embodiment, the RAGE polypeptide comprises consecutive amino acids corresponding to amino acids 24-226 of human RAGE (SEQ ID NO: 11). In one embodiment, the RAGE polypeptide comprises consecutive amino acids corresponding to amino acids 24-339 of human RAGE (SEQ ID NO: 4).

In another embodiment, the antagonist comprises a RAGE fusion protein and a pharmaceutically acceptable carrier, wherein the RAGE fusion protein comprises a RAGE polypeptide linked to a second, non-RAGE polypeptide wherein the RAGE polypeptide comprises a RAGE ligand binding site. In one embodiment, the RAGE polypeptide is linked to a polypeptide comprising an immunoglobulin domain or a portion of an immunoglobulin domain. In one embodiment, the polypeptide comprising an immunoglobulin domain comprises at least a portion of at least one of the CH2 or the C_{H}3 domains of a human IgG. In one embodiment, the RAGE ligand binding site comprises consecutive amino acids comprising the sequence A-Q-N-I-T-A-R-I-G-E-P-L-V-L-K-C-K-G-A-P-K-K-P-P-Q-R-L-E-W-K (SEQ ID NO. 6) or a sequence 90% identical thereto or Q-N-I-T-A-R-I-G-E-P-L-V-L-K-C-K-G-A-P-K-K-P-P-Q-R-L-E-W-K (SEQ ID NO. 8) or a sequence 90% identical thereto. In one embodiment, the RAGE polypeptide comprises consecutive amino acids corresponding to amino acids 24-116 of human RAGE (SEQ ID NO: 9).

Other RAGE fusion proteins are also described, for example, in the following publications: PCT International Application Publication No. WO/2004/016229, PCT International Application Publication No. WO 2006/017647 A1, PCT International Application Publication No. WO 2006/017643 A1, U.S. Patent Application Publication No. US 2006/140933, U.S. Patent Application Publication No. US 2006/078562, U.S. Patent Application No. US 2006/0057679, U.S. Patent Application Publication No. 2006/0030527, all of which are hereby incorporated by reference. It is understood that these are non-limiting examples of RAGE fusion proteins.

In another embodiment, the antagonist is a small molecule. In one embodiment, the small molecule is a compound having the structure:

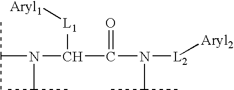

wherein L₁ is a C₁-C₄ alkyl group and L₂ is a direct bond, and Aryl₁ and Aryl₂ are aryl, wherein each of Aryl₁ and Aryl₂ are substituted by at least one lipophilic group selected from the group consisting of
a) —Y—C₁₋₆ alkyl;
b) —Y-aryl;
c) —Y—C₁₋₆ alkylaryl;
d) —Y—C₁₋₆ alkyl-NR₇R₈;
e) —Y—C₁₋₆ alkyl-W—R₂₀;
   wherein
Y and W are, independently selected from the group consisting of —CH₂—, —O—, —N(H), —S—, SO₂—, —CON(H)—, —NHC(O)—, —NHCON(H)—, —NHSO₂—, —SO₂(H)—, —C(O)—O—, —NHSO₂NH—, —O—CO—,

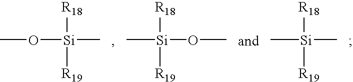

and
f) halogen, hydroxyl, cyano, carbamoyl, and carboxyl;
   wherein
R₁₈ and R₁₉ are independently selected from the group consisting of aryl, C₁-C₆ alkyl, C₁-C₈ alkylaryl, C₁-C₆ alkoxy, and C₁-C₆ alkoxyaryl;
R₂₀ is selected from the group consisting of aryl, C₁-C₆ alkyl, and C₁-C₆ alkylaryl;

R₇, R₈, R₉ and R₁₀ are independently selected from the group consisting of hydrogen, aryl, C₁-C₆ alkyl, and C₁-C₆ alkylaryl; and wherein R₇ and R₈ may be taken together to form a ring having the formula —(CH₂)ₘ—X—(CH₂)ₙ— bonded to the nitrogen atom to which R₇ and R₈ are attached, wherein m and n are, independently, 1, 2, 3, or 4; X is selected from the group consisting of —CH₂—, —S(O₂)—, —C(O)—, —CON(H)—, —NHC(O)—, —NHCON(H)—, —NHSO₂—, —SO₂N(H)—, —C(O)—O—, —O—C(O)—, —NHSO₂NH—,

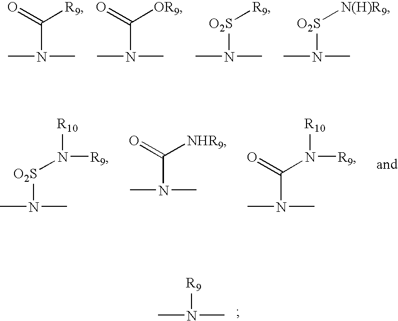

or a pharmaceutically acceptable salt thereof, wherein at least one of Aryl₁ and Aryl₂ is substituted with a lipophilic group of the formula —Y—C₁₋₆-alkyl-NR₇R₈.

In one embodiment, the small molecule is a compound having the structure:

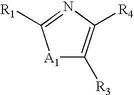

wherein
R₁ is -hydrogen, -alkyl, -alkenyl, or -alkynyl, A₁ is —N(R₂)—;
wherein
R₂ is -phenyl,
R₃ is
a) -hydrogen,
b) -halogen,
c) -hydroxyl,
d) -cyano,
e) -carbamoyl,
f) -carboxyl,
g) -aryl,
h) -cycloalkyl,
i) -alkyl,
j) -alkenyl,
k) -alkynyl,
l) -alkylene-aryl,
m) -alkylene-cycloalkyl,
n) -fused cycloalkylaryl,
o) -alkylene-fused cycloalkylaryl,
p) —C(O)—O-alkyl,
q) —C(O)—O-alkylene-aryl,
r) —C(O)—NH-alkyl,
s) —C(O)—NH-alkylene-aryl,
t) —SO₂-alkyl,
u) —SO₂-alkylene-aryl,
v) —SO₂-aryl,
w) —SO₂—NH-alkyl,
x) —SO₂NH-alkylene-aryl,
y) —C(O)-alkyl,
z) —C(O)-alkylene-aryl,
aa) -G₄-G₅-G₆-R₇,
bb) —Y₁-alkyl,
cc) —Y₁-aryl,
dd) —Y₁-alkylene-aryl,
ee) —Y₁-alkylene-NR₉R₁₀, or
ff) —Y₁-alkylene-W₁—R₁₁,
wherein
   G₄ and G₆ are independently selected from the group consisting of: alkylene, alkenylene, alkynylene, cycloalkylene, arylene, -alkylene-aryl, alkenylene-aryl, -alkenylene-heteroaryl, and a direct bond;
   G₅ is —O—, —S—, —N(R₈)—, —S(O)—, —S(O)₂—, —C(O)—, —O—C(O)—, —C(O)—O—, —C(O)N(R₈)—, —N(R₈)C(O)—, —S(O)₂N(R₈)—, N(R₈)S(O)₂—, —O-alkylene-C(O)—, —(O)C-alkylene-O—, —O-alkylene-, -alkylene-O—, alkylene, alkenylene, alkynylene, cycloalkylene, arylene, fused cycloalkylarylene, or a direct bond, wherein R₈ is -hydrogen, -aryl, -alkyl, -alkylene-aryl, or -alkylene-O-aryl;
wherein
R₇ is -hydrogen, -aryl, -cycloalkyl, -alkyl, -alkenyl, -alkynyl, -alkylene-aryl, -alkylene-cycloalkyl, -fused cycloalkylaryl, or -alkylene-fused cycloalkylaryl;
Y₁ and W₁ are independently selected from the group consisting of —CH₂—, —N(H), SO₂—, —CON(H)—, —NHC(O)—, —NHCON(H)—, —NHSO₂—, —SO₂N(H)—C(O)—O—, —NHSO₂NH—, —O—CO—,

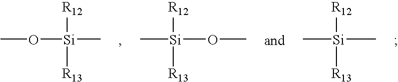

wherein
R₁₂ and R₁₃ are independently selected from the group consisting of: -aryl, -alkyl, -alkylene-aryl, -alkoxy, and -alkylene-O-aryl; and R₉, R₁₀, and R¹² are independently selected from the group consisting of: -aryl, -alkyl, and -alkylene-aryl;
R₄ is
a) -phenyl,
b) -phenylene-G₅-G₈-R₇,
c) -phenylene-alkylene-G₉-G₆-R₇, or
d) -phenylene-alkenylene-G₉-G₆-R₇, wherein
G₆ is alkylene, alkenylene, alkynylene, cycloalkylene, heterocyclylene, arylene, heteroarylene, -alkylene-aryl, -alkylene-heteroaryl, -alkenylene-aryl, -alkenylene-heteroaryl, or a direct bond;
G₅ is —O—, —N(R₈)—, —S(O)—, —S(O)₂—, —C(O)—, —O—C(O)—, —C(O)—O—, —C(O)N(R₈)—, N(R₈)C(O)—, —S(O)₂N(R₈)—, N(R₈)S(O)₂—, —O-alkylene-C(O)—, —(O)C-alkylene-O—, —O-alkylene-, -alkylene-O—, alkylene, alkenylene, alkynylene, cycloalkylene, heterocyclylene, arylene, heteroarylene, fused cycloalkylarylene, fused cycloalkylheteroarylene, fused heterocyclylarylene, fused heterocyclylheteroarylene, or a direct bond,
   wherein
R₈ is -hydrogen, -aryl, -alkyl, -alkylene-aryl, or -alkylene-O-aryl; R₇ is hydrogen, aryl, heteroaryl, cycloalkyl, heterocyclyl, alkyl, alkenyl, alkynyl, -alkylene-aryl, -alkylene-heteroaryl, -alkylene-heterocyclyl, -alkylene-cycloalkyl, fused cycloalkylaryl, fused cycloalkylheteroaryl, fused heterocyclylaryl, fused heterocyclylheteroaryl, alkylene-fused cycloalkylaryl, -alkylene-fused cycloalkylheteroaryl, -alkylene-fused heterocyclylaryl, or -alkylene-fused heterocyclylheteroaryl;
   wherein
the aryl and/or alkyl group(s) in R₃, R₇, R₈, R₉, R₁₀, R₁₁, R₁₂, and R₁₃, may be optionally substituted 1-4 times with a substituent group, wherein said substituent group(s) are independently selected from the group consisting of:
   a) —H,
   b) -halogen,
   c) -hydroxyl,
   d) -cyano,
   e) -carbamoyl,
   f) -carboxyl,
   g) —Y₂-alkyl,
   h) —Y₂-aryl,
   i) —Y₂-alkylene-aryl,
   j) —Y₂-alkylene-W₂—R₁₈,
   k) —Y₃—Y₄—NR₂₃R₂₄)
   l) —Y₃—Y₄—NH—C(═NR₂₅)NR₂₃R₂₄, and
   m) —Y₃—Y₄—C(═NR₂₅)NR₂₃R₂₄
      wherein
Y₂ and W₂ are independently selected from the group consisting of —CH₂—, —O—, —N(H), —S—, SO₂—, —CON(H)—, —NHC(O)—, —NHCON(H)—, —NHSO₂—, —SO2N(H)—, —C(O)—O—, —NHSO₂NH—, —O—S(O)₂—, —O—CO—,

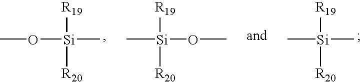

wherein R₁₉ and R₂₀ are independently selected from the group consisting of: -hydrogen, -aryl, -alkyl, -alkylene-aryl, -alkoxy, and -alkylene-O-aryl; R₁₃ is -aryl, alkyl, -alkylene-aryl, or -alkylene-O-aryl;
Y₃ is selected from the group consisting of a direct bond, —CH₂—, —O—, —N(H), —S—, SO₂—, —C(O)—, —CON(H)—, —NHC(O)—, —NHCON(H)—, —NHSO₂—, —SO₂N(H)—, —C(O)—O—, —NHSO₂NH—, —O—CO—,

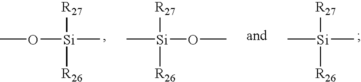

wherein R₂₇ and R₂₆ are independently selected from the group consisting of: -aryl, -alkyl, -alkylene-aryl, -alkoxy, and -alkyl-O-aryl;
Y₄ is
   a) -alkylene,
   b) -alkenylene,
   c) -alkynylene,
   d) -arylene,
   e) -cycloalkylene,
   f) -alkylene-arylene,
   g) -alkylene-cycloalkylene,
   h) -arylene-alkylene,
   i) -cycloalkylene-alkylene,
   j) —O—,
   k) —S—,
   l) —S(O)₂—, or
   m) —S(O)—,
   wherein said alkylene groups may optionally contain one or more O, S, S(O), or SO₂ atoms;
   and R₂₃, R₂₄, and R₂₅ are independently selected from the group consisting of: -hydrogen, -aryl, -alkyl, -alkylene-aryl, and -alkylene-O-aryl, and
   wherein
   R₂ may be optionally substituted 1-4 times with a substituent group, wherein said substituent group(s) are independently selected from the group consisting of:
      a) —H,
      b) -halogen,
      c) -hydroxyl,
      d) -cyano,
      e) -carbamoyl,
      f) -carboxyl,
      g) —Y₂-alkyl,
      h) —Y₂-aryl,
      i) —Y₂-heteroaryl,
      j) —Y₂-alkylene-heteroaryl-aryl,
      k) —Y₂-alkylene-aryl,
      l) —Y₂-alkylene-W₂—R₁₅,
      m) —Y₃—Y₄—NR₂₃R₂₄
      n) —Y₃—Y₄—NH—C(═NR₂₅)NR₂₃R₂₄,
      j) —Y₃—Y₄—C(═NR₂₅)NR₂₃R₂₄, and
      k) —Y₃—Y₄—Y₅-A₂,
      wherein
         Y₂ and W₂ are independently selected from the group consisting of —CH₂—, —O—, —N(H), —S—, SO₂—, —CON(H)—, —NHC(O)—, —NHCON(H)—, —NHSO₂—, —SO₂N(H)—, —C(O)—O—, —NHSO₂NH—, —O—S(O)₂—, —O—CO—,

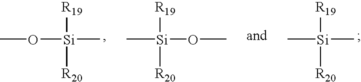

wherein R₁₉ and R₂₀ are independently selected from the group consisting of: -hydrogen, -aryl, -alkyl, -alkylene-aryl, alkoxy, and -alkylene-O-aryl;
R₁₈ is -aryl, -alkyl, -alkylene-aryl, -alkylene-heteroaryl, or -alkylene-O-aryl;
Y₃ and Y₅ are independently selected from the group consisting of a direct bond, —CH₂—, —O—, —N(H), —S—, SO₂—, —C(O)—, —CON(H)—, —NHC(O)—, —NHCON(H)—, —NHSO₂—, —SO₂N(H)—, —C(O)—O—, —NHSO₂NH—, —O—CO—,

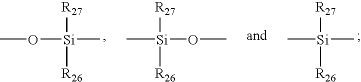

wherein R₂₇ and R₂₆ are independently selected from the group consisting of: -aryl, -alkyl, -alkylene-aryl, -alkoxy, and -alkyl-O-aryl;
Y₄ is
   a) -alkylene,
   b) -alkenylene,
   c) -alkynylene,
   d) -arylene,
   e) -heteroarylene,
   f) -cycloalkylene,
   g) -heterocyclylene,
   h) -alkylene-arylene,
   i) -alkylene-heteroarylene,
   j) -alkylene-cycloalkylene,
   k) -alkylene-heterocyclylene,
   l)-arylene-alkylene,
   m) -heteroarylene-alkylene,
   n) -cycloalkylene-alkylene,
   o) -heterocyclylene-alkylene,
   p) —O—,
   q) —S—,
   r) —S(O)₂—, or
   s) —S(O)—,
      wherein said alkylene groups may optionally contain one or more O, S, S(O), or SO₂ atoms;
A₂ is
   a) heterocyclyl, fused arylheterocyclyl, or fused heteroarylheterocyclyl, containing at least one basic nitrogen atom, or
   b) -imidazolyl, and
      R₂₃, R₂₄, and R₂₅ are independently selected from the group consisting of: -hydrogen, -aryl, -heteroaryl, -alkylene-heteroaryl, -alkyl, alkylene-aryl, -alkylene-O-aryl, and -alkylene-O-heteroaryl; and R₂₃ and R₂₄ may be taken together to form a five-membered ring having the formula —(CH₂)ₛ—X₃—(CH₂)ₜ— bonded to the nitrogen atom to which R₂₃ and R₂₄ are attached
      wherein
      s and t are, independently, 1, 2, 3, or 4;
      X₃ is a direct bond, —CH₂—, —O—, —S—, —S(O)₂—, —C(O)—, —CON(H)—, —NHC(O)—, —NHCON(H)—, —NHSO₂—, —SO₂N(H)—, —C(O)—O—, —O—C(O)—, —NHSO₂NH—,

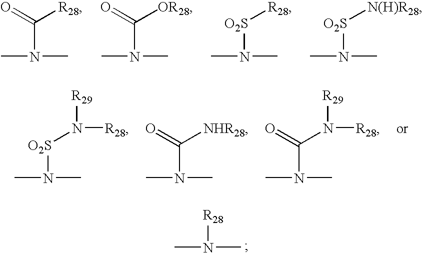

wherein R₂₈ and R₂₉ are independently selected from the group consisting of: -hydrogen, -aryl, -heteroaryl, -alkyl, -alkylene-aryl, and -alkylene-heteroaryl;
wherein the alkyl and/or aryl groups in the optional substituents
   g) —Y₂-alkyl,
   h) —Y₂-aryl,
   i) —Y₂-heteroaryl,
   j) —Y₂-alkylene-heteroaryl,
   k) —Y₂-alkylene-aryl,
   l) —Y₂-alkylene-W₂—R₁₈,
   m) —Y₃—Y₄—NR₂₃R₂₄,
   n) —Y₃—Y₄—NH—C(═NR₂₅)NR₂₃R₂₄,
   o) —Y₃—Y₄—C(═NR₂₅)NR₂₃R₂₄, and
   p) —Y₃—Y₄—Y₅-A₂,
of R₂ may be optionally substituted 1-4 times with a substituent independently selected from the group consisting of:
   a) halogen,
   b) perhaloalkyl,
   c) alkyl,
   d) cyano,
   e) alkyloxy,
   f) aryl, and
   g) aryloxy, and
wherein the aryl and/or alkyl group(s) in R₄ may be optionally substituted 1-4 times with a substituent group, wherein said substituent group(s) are independently selected from the group consisting of:
   a) —H,
   b) -halogen,
   c) -hydroxyl,
   d) -cyano,
   e) -carbamoyl,
   f) -carboxyl,
   g) —Y₂-alkyl,
   h) —Y₂-aryl,
   i) —Y₂-heteroaryl,
   j) —Y₂-alkylene-heteroaryl-aryl,
   k) —Y₂-alkylene-aryl,
   l) —Y₂-alkylene-W₂—R₁₈,
   m) —Y₃—Y₄—NR₂₃R₂₄,
   n) —Y₃—Y₄—NH—C(═NR₂₅)NR₂₃R₂₄,
   o) —Y₃—Y₄—C(═NR₂₅)NR₂₃R₂₄, and
   p) —Y₃—Y₄—Y₅-A₂,
wherein
   Y₂ and W₂ are independently selected from the group consisting of —CH₂—, —O—, —N(H), —S—, SO₂—, —CON(H)—, —NHC(O)—, —NHCON(H)—, —NHSO₂—, —SO₂N(H)—, —C(O)—O—, —NHSO₂NH—, —O—S(O)₂—, —O—CO—,

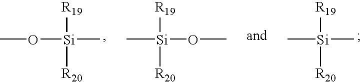

wherein R₁₉ and R²⁰ are independently selected from the group consisting of: -hydrogen, -aryl, -alkyl, -alkylene-aryl, alkoxy, and -alkylene-O-aryl;
R₁₈ is -aryl, -alkyl, -alkylene-aryl, -alkylene-heteroaryl, or -alkylene-O-aryl;
Y₃ and Y₅ are independently selected from the group consisting of a direct bond, —CH₂—, —O—, —N(H), —S—, SO₂—, —C(O)—, —CON(H)—, —NHC(O)—, —NHCON(H)—, —NHSO₂—, —SO₂N(H)—, —C(O)—O—, —NHSO₂NH—, —O—CO—,

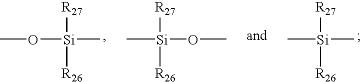

wherein R₂/and R₂₆ are independently selected from the group consisting of: -aryl, -alkyl, -alkylene-aryl, -alkoxy, and -alkyl-O-aryl;
Y₄ is
a) -alkylene,
b) -alkenylene,
c) -alkynylene,
d) -arylene,
e) -heteroarylene,
f) -cycloalkylene,
g) -heterocyclylene,
h) -alkylene-arylene,
i) -alkylene-heteroarylene,
j) -alkylene-cycloalkylene,
k) -alkylene-heterocyclylene,
l) -arylene-alkylene,
m) -heteroarylene-alkylene,
n) -cycloalkylene-alkylene,
o) -heterocyclylene-alkylene,
p) —O—,
q) —S—,
r) —S(O)₂—, or
s) —S(O)—,
wherein said alkylene groups may optionally contain one or more O, S, S(O), or SO₂ atoms;
A₂ is
   a) heterocyclyl, fused arylheterocyclyl, or fused heteroarylheterocyclyl, containing at least one basic nitrogen atom, or
   b) -imidazolyl, and
R₂₃, R₂₄, and R₂₅ are independently selected from the group consisting of: -hydrogen, -aryl, -heteroaryl, -alkylene-heteroaryl, -alkyl, alkylene-aryl, -alkylene-O-aryl, and alkylene-O-heteroaryl; and R₂₃ and R₂₄ may be taken together to form a five-membered ring having the formula —(CH₂)ₛ—X₃—(CH₂)ₜ— bonded to the nitrogen atom to which R₂₃ and R₂₄ are attached
   wherein
   s and t are, independently, 1, 2, 3, or 4;
   X₃ is a direct bond, —CH2-, —O—, —S—, —S(O)₂—, —C(O)—, —CON(H)—, —NHC(O)—, —NHCON(H)—, —NHSO₂—, —SO₂N(H)—, —C(O)—O—, —O—C(O)—, —NHSO₂NH—,

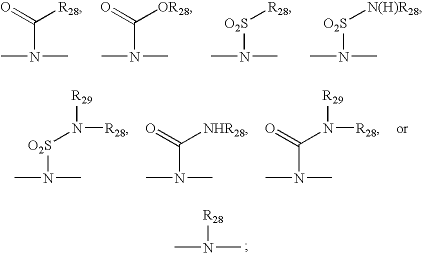

wherein R₂₈ and R₂₉ are independently selected from the group consisting of: -hydrogen, -aryl, -heteroaryl, -alkyl, -alkylene-aryl, and -alkylene-heteroaryl;
wherein the alkyl and/or aryl groups in the optional substituents
   g) —Y₂-alkyl,
   h) —Y₂-aryl,
   i) —Y₂-heteroaryl,
   j) —Y₂-alkylene-heteroaryl,
   k) —Y₂-alkylene-aryl,
   l) —Y₂-alkylene-W₂—R₁₈,
   m) —Y₃—Y₄—NR₂₃R₂₄,
   n) —Y₃—Y₄—NH—C(═NR₂₅)NR₂₃R₂₄,
   o) —Y₃—Y₄—C(═NR₂₅)NR₂₃R₂₄, and
   p) —Y₃—Y₄—Y₅-A₂,
   of R₂ and R₄ may be optionally substituted 1-4 times with a substituent independently selected from the group consisting of:
      a) halogen,
      b) perhaloalkyl,
      c) alkyl,
      d) cyano,
      e) alkyloxy,
      f) aryl, and
      g) aryloxy, and
wherein the ring or rings containing a heteroatom in the heteroaryl, heteroarylene, heterocyclyl, heterocyclene, fused arylheterocyclyl, or fused heteroarylheterocyclyl groups in R₂ or R₄ or in a substituent of R₂ or R₄ is a five membered nitrogen containing ring, and
wherein
   at least one of R₂ and R₄ is substituted with at least one group of the formula
      —Y₃—Y₄—NR₂₃R₂₄,
      —Y₃—Y₄—NH—C(═NR₂₅)NR₂₃R₂₄,
      —Y₃—Y₄(═NR₂₅)NR₂₃R₂₄, or
      —Y₃—Y₄—Y₅-A₂,
   with the proviso that no more than one of R₂₃, R₂₄, and R₂₅ is aryl or heteroaryl;
   or a pharmaceutically acceptable salt thereof, and a pharmaceutically acceptable carrier.

In one embodiment, the antagonist is a compound having the structure

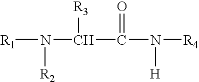

wherein
   R₁ and R₂ are independently selected from
      a) —H;
      b) alkyl;
      c) -aryl;
      d) alkylaryl;
      e) —C(O)—O—C₁₋₄ alkyl;
      f) alkylaryl;
      h) alkylaryl;
      i) alkyl;
      j) alkylaryl;
      k) —SO₂-aryl;
      l) —SO₂—NH—C₁₋₆ alkyl;
      m) alkylaryl;
      n)

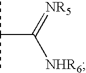

o) alkyl; and
p) —C(O)—C₁₋₆ alkylaryl;
R₃ is selected from
   (a) -aryl; and
   (b) —C₁₋₃ alkylaryl,
   wherein aryl is substituted by C₁₋₆ alkyl, C₁₋₆ alkoxy, C₁₋₆ alkylaryl, or C₁₋₆ alkoxyaryl;
R₄ is selected from

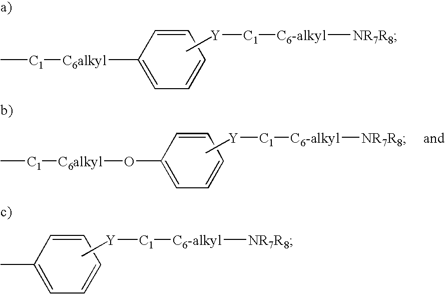

R₅ and R₆ are independently selected from the group consisting of hydrogen, C₁-C₆ alkyl, C₁-C₆ alkylaryl, and aryl; and wherein
the aryl and/or alkyl group(s) in R₁, R₂, R₄, R₅, R₆, R₇, R₈, R₉, R₁₀, R₁₈, R₁₉, and R₂₀ may be optionally substituted 1-4 times with a substituent group, wherein said substituent group(s) or the term substituted refers to groups selected from the group consisting of:
   a) —H;
   b) alkyl; —
   —Y-aryl; —
   —Y—C₁₋₆ alkylaryl;
   —Y—C₁₋₆ alkyl-NR₇R₈; and
   —Y—C₁₋₆-alkyl-W—R₂₀; and
   c) halogen, hydroxyl, cyano, carbamoyl, or carboxyl; and
wherein
   Y and W are independently selected from the group consisting of —CH₂—O—, —N(H), —S—, SO₂—, —CON(H)—, —NHC(O)—, —NHCON(H)—, —NHSO₂—, —SO₂N(H)—, —C(O)—O—, —NHSO₂NH—, —O—CO—,

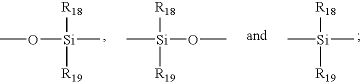

R₁₈ and R₁₉ are independently selected from the group consisting of aryl, C1-C₆ alkyl, C1-C₆ alkylaryl, C1-C₆ alkoxy, and C1-C₆ alkoxyaryl;
R₂₀ is selected from the group consisting of aryl, C1-C₆ alkyl, and C1-C₆ alkylaryl;
R₇, R₈, R₉ and R₁₀ are independently selected from the group consisting of hydrogen, aryl C1-C₆ alkyl, and C1-C₆ alkylaryl; and wherein
R₇ and R₈ may be taken together to form a ring having the formula —(CH₂)ₘ—X—(CH₂)ₙ— bonded to the nitrogen atom to which R₇ and R₈ are attached, and/or R₅ and R₆ may, independently, be taken together to form a ring having the formula —(CH₂)ₘ—X—(CH₂)ₙ— bonded to the nitrogen atoms to which R₅ and R₆ are attached, wherein m and n are, independently, 1, 2, 3, or 4; X is selected from the group consisting of —CH₂—, —O—, —S—, —S(O₂)—, —C(O)—, —CON(H)—, —NHC(O)—, —NHCON(H)—, —NHSO₂—, —SO₂N(H)—, —C(O)—O—, —O—C(O)—, —NHSO₂NH—,

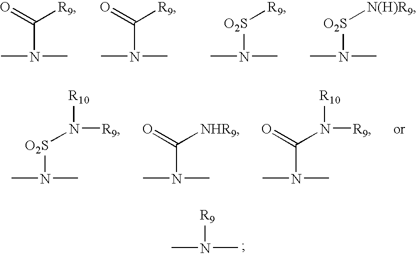

or a pharmaceutically acceptable salt thereof.

In another embodiment, the small molecule has the structure:

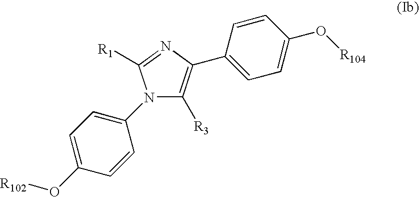

wherein
R₁ is -hydrogen, -alkyl, or -alkenyl,
R₃ is -hydrogen or -alkyl; and
R₁₀₂ and R₁₀₄ are independently selected from the group consisting of:
   a) —H;
   b) -alkyl;
   c) -aryl;
   d) -heteroaryl;
   e) -alkylene-heteroarylene-aryl;
   f) -alkylene-aryl;
   g) -alkylene-W₂—R₈;
   h) —Y₄—NR₂₃R₂₄;
   i) —Y₄—NH—C(═NR₂₅)NR₂₃R₂₄;
   j) —Y₄—C(═NR₂₅)NR₂₃R₂₄; and
   k) —Y₄—Y₅-A₂;
      wherein
         W₂ is —CH₂—, —O—, —N(H), —S—, SO₂—, —CON(H)—, —NHC(O)—, —NHCON(H)—, —NHSO₂—, —SO₂N(H)—, —C(O)—O—, —NHSO₂NH—, —O—S(O)₂—, —O—CO—,

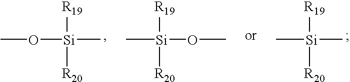

wherein R₁₉ and R₂₀ are independently selected from the group consisting of: -hydrogen, -aryl, -alkyl, -alkylene-aryl, alkoxy, and -alkylene-O-aryl;
R₁₈ is -aryl, -alkyl, -alkylene-aryl, -alkylene-heteroaryl, or -alkylene-O-aryl;
Y₅ is a direct bond, —CH₂—, —O—, —N(H), —S—, SO₂—, —C(O)—, —CON(H)—, —NHC(O)—, —NHCON(H)—, —NHSO₂—, —SO₂N(H)—, —C(O)—O—, —NHSO₂NH—, —O—CO—,

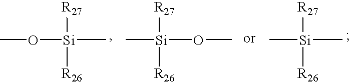

wherein R₂₇ and R₂₆ are independently selected from the group consisting of -aryl, -alkyl, -alkylene-aryl, alkoxy, and -alkyl-O-aryl;
Y₄ is
a) -alkylene;
b) -alkenylene;
c) -alkynylene;
d) -arylene;
e) -heteroarylene;
f) -cycloalkylene;
g) -heterocyclylene;
h) -alkylene-arylene;
i) -alkylene-heteroarylene;
j) -alkylene-cycloalkylene;
k) -alkylene-heterocyclylene;
l) -arylene-alkylene;
m) -heteroarylene-alkylene;
n) -cycloalkylene-alkylene;
o) -heterocyclylene-alkylene;
p) —S(O)₂—; or
q) —S(O)—;
wherein said alkylene groups may optionally contain one or more O, S, S(O), or SO₂ atoms;
A₂ is
a) heterocyclyl, fused arylheterocyclyl, or fused heteroarylheterocyclyl, containing at least one basic nitrogen atom, or
b) -imidazolyl,
R₂₃, R₂₄, and R₂₅ are independently selected from the group consisting of: -hydrogen, -aryl, -heteroaryl, -alkylene-heteroaryl, alkyl, -alkylene-aryl, -alkylene-O-aryl, and -alkylene-O-heteroaryl; and R₂₃ and R₂₄ may be taken together to form a five membered ring having the formula —(CH₂)ₛ—X₃—(CH₂)ₜ— bonded to the nitrogen atom to which R₂₃ and R₂₄ are attached
wherein
s and t are, independently, 1, 2, 3, or 4;
X₃ is a direct bond, —CH₂—, —O—, —S—, —S(O)₂—, C(O)—, —CON(H)—, —NHC(O)—, —NHCON(H)—, —NHSO₂—, —SO₂N(H)—, —C(O)—O—, —O—C(O)—, —NHSO₂NH—,

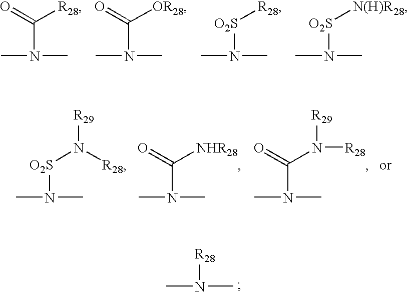

wherein R₂₈ and R₂₉ are independently selected from the group consisting of: -hydrogen, -aryl, -heteroaryl, -alkyl, -alkylene-aryl, and -alkylene-heteroaryl;
wherein
   the alkyl, alkylene, alkenyl, heteroaryl, heteroarylene, cycloalkylene, heterocyclylene, arylene, fused arylheterocyclyl, fused heteroarylheterocyclyl, and/or aryl groups of R₁, R₃, R₂₃, R₂₄, R₂₅, A₂, Y₄R₁₀₂ and R₁₀₄ may be optionally substituted 1-4 times with a substituent group independently selected from the group consisting of:
      a) halogen;
      b) haloalkyl;
      c) alkyl;
      d) cyano;
      e) alkyloxy;
      f) aryl; and
      g) aryloxy
   wherein
      at least one of R₁₀₂ and R₁₀₄ is a group of the formula
         —Y₄—NR₂₃R₂₄,
         —Y₄—NH—C(═NR₂₅)NR₂₃R₂₄,
         —Y₄—C(═NR₂₅)NR₂₃R₂₄, or
         —Y₄—Y₅-A₂,
         with the proviso that no more than one of R₂₃, R₂₄, and R₂₅ is aryl or heteroaryl;
            or a pharmaceutically acceptable salt thereof.

In another embodiment, the small molecule has the structure:

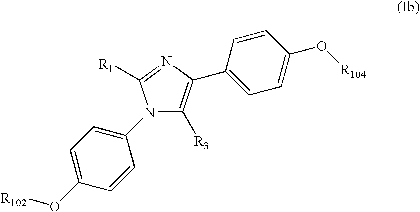

wherein R₁ is a hydrogen, methyl, ethyl, propyl, butyl, iso-butyl, 3-butenyl, tert-butyl, 2,4,4-trimethyl-pentyl, 1-ethyl-propyl, or 1-propyl-butyl, and R₃ is -hydrogen,
or a pharmaceutically acceptable salt thereof.

In another embodiment, the small molecule has the structure,

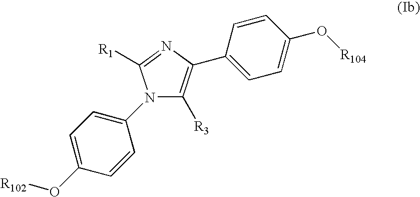

wherein R₁₀₂ and R₁₀₄ are independently selected from the group consisting of:
2-(4-chlorophenyl)-ethyl,
3-(N,N′-diethylamino)-propyl,
2-amino-ethyl,
2-(guanidinyl)-ethyl,
3-(N,N′-dimethylamino)-propyl,
3-fluoro-4-trifluoromethyl-phenyl,
4-fluoro-3-trifluoromethyl-phenyl,
4-phenyl-phenyl,
4-trifluoromethyl-benzyl,
3,4-dichloro-phenyl,
2,4-dichloro-phenyl,
benzyl,
4-phenoxy-benzyl,
3,4,5-trimethoxybenzyl,
2-(pyrrolidin-1-yl)-ethyl,
2,2′-dimethyl-3-(N,N′-diethylamino)-propyl,
2-(N,N′-diisopropylamino)-ethyl,
4-bromo-benzyl,
4-chlorophenyl,
3,3-diphenylpropyl,
2-(biphenyl-4-yl)-acetamido
2-(9H-carbazole)-ethyl,
4-methoxyphenyl,
4-tert-butyl-phenyl, and
naphthylen-2-ylmethyl,
   or a pharmaceutically acceptable salt thereof.

In another embodiment, the small molecule has the structure:

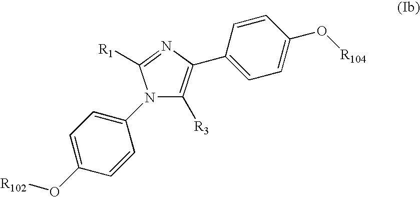

wherein
R₁ is -alkyl,
R₃ is hydrogen;
R₁₀₂ is -aryl or -alkylene-aryl substituted with at least one of a halogen, a haloalkyl, or an alkoxy group; and
R₁₀₄ is —Y₄—NR₂₃R₂₄ or —Y₄—Y₅-A₂,
   or a pharmaceutically acceptable salt thereof.

In another embodiment, the small molecule has the structure:

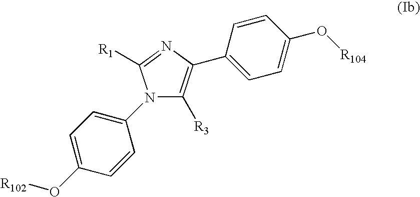

wherein
R₃ is hydrogen; and
R₁₀₂ and R₁₀₄ are independently selected from the group consisting of -aryl and -alkylene-aryl,
   wherein
the alkyl, alkylene, or aryl groups of R₁₀₂ and R₁₀₄ are optionally substituted with at least one of a halogen, a haloalkyl, or an alkoxy group, and wherein at least one of R₁₀₂ and R₁₀₄ is —Y₄—NR₂₃R₂₄ or —Y₄—Y₅-A₂, wherein Y₄ is alkylene,
   or a pharmaceutically acceptable salt thereof.

In another embodiment, the small molecule is selected from the group consisting of:
(1) {3-[4-(2-butyl-4-{4-[2-(4-chloro-phenyl)-ethoxy]-phenyl}-imidazol-1-yl)-phenoxy]-propyl}-diethyl-amine;
(2) {3-[4-(4-{4-[2-(4-chloro-phenyl)-ethoxy]-phenyl}-2-isobutyl-imidazol-1-yl)-phenoxy]-propyl}-diethyl-amine;
(3) [3-(4-{2-butyl-1-[4-(4-chloro-phenoxy)-phenyl]-1H-imidazol-4-yl}-phenoxy)-propyl]-diethyl-amine;
(4) 3-(4-{2-butyl-1-[4-(4-fluoro-3-trifluoromethyl-phenoxy)-phenyl]-1H-imidazol-4-yl}-phenoxy)-propyl]-diethyl-amine;
(5) diethyl-[3-(4-{1-[4-(4-fluoro-3-trifluoromethyl-phenoxy)-phenyl]-2-methyl-1H-imidazol-4-yl}-phenoxy)-propyl]-amine;
(6) [3-(4-{2-butyl-1-[4-(3-tert-butyl-phenoxy)-phenyl]-1H-imidazol-4-yl}-phenoxy)-propyl]-diethyl-amine;
(7) (3-{4-[4-{4-[2-(4-chloro-phenyl)-ethoxy]-phenyl}-2-(1-ethyl-propyl)-imidazol-1-yl]-phenoxy}-propyl)-diethyl-amine;
(8) {3-[4-(4-{4-[2-(4-chloro-phenyl)-ethoxy]-phenyl}-2-isobutyl-5-methyl-imidazol-1-yl)-phenoxy]-propyl}-diethyl-amine;
(9) {3-[4-(4-{4-[2-(4-chloro-phenyl)-ethoxy]-phenyl}-2-isobutyl-5-propyl-imidazol-1-yl)-phenoxy]-propyl}-diethyl-amine;
(10) {3-[4-(5-butyl-4-{4-[2-(4-chloro-phenyl)-ethoxy]-phenyl}-2-isobutyl-imidazol-1-yl)-phenoxy]-propyl}-diethyl-amine;
(11) [3-(4-{1-[4-(4-chloro-phenoxy)-phenyl]-2-isobutyl-1H-imidazol-4-yl}-phenoxy)-propyl]-diethyl-amine;
(12) [3-(4-{2-butyl-1-[4-(4-chloro-phenoxy)-phenyl]-5-methyl-1H-imidazol-4-yl}-phenoxy)-propyl]-diethyl-amine;
(13) [3-(4-{2-butyl-1-[4-(4-chloro-phenoxy)-phenyl]-5-propyl-1H-imidazol-4-yl}-phenoxy)-propyl]-diethyl-amine;
(14) [3-(4-{2,5-dibutyl-1-[4-(4-chloro-phenoxy)-phenyl]-1H-imidazol-4-yl}-phenoxy)-propyl]-diethyl-amine;
(15) 2-butyl-1-[4-(4-chloro-phenoxy)-phenyl]-4-[4-(2-pyrrolidin-1-yl-ethoxy)-phenyl]-1H-imidazole;
(16) [3-(4-{2-butyl-4-[4-(4-chloro-phenoxy)-phenyl]-imidazol-1-yl}-phenoxy)-propyl]-dimethyl-amine;
(17) (3-{4-[2-butyl-1-(4-p-tolyloxy-phenyl)-1H-imidazol-4-yl]-phenoxy}-propyl)-diethyl-amine;
(18) [3-(4-{2-butyl-1-[4-(4-fluoro-phenoxy)-phenyl]-1H-imidazol-4-yl}-phenoxy)-propyl]-diethyl-amine;
(19) [3-(4-{4-[4-(3,3-diphenyl-propoxy)-phenyl]-2-isobutyl-imidazol-1-yl}-phenoxy)-propyl]-diethyl-amine, and
   pharmaceutically acceptable salts thereof.

Other RAGE antagonists are described, for example, in the following publications: U.S. Patent Application Publication No. US 2008/119512, U.S. Pat. No. 7,361,678, PCT International Application Publication No. WO 2007/089616, PCT International Application Publication No. WO 2007/076200, PCT International Application Publication No. WO 2007/0286858, all of which are hereby incorporated by reference. It is understood that these are non-limiting examples of RAGE antagonists.

This invention further provides a method for treating hyperglycemia in a subject which comprises administering to the subject an antagonist of a receptor for advanced glycation end products (RAGE) in an amount effective to inhibit binding of a ligand of RAGE to RAGE so as to thereby treat hyperglycemia in the subject.

This invention further provides a method for reducing levels of cholesterol in a subject which comprises administering to the subject an antagonist of a receptor for advanced glycation end products (RAGE) in an amount effective to inhibit binding of a ligand of RAGE to RAGE so as to thereby reduce cholesterol levels in the subject.

This invention further provides a method for reducing levels of insulin in a subject which comprises administering to the subject an antagonist of a receptor for advanced glycation end products (RAGE) in an amount effective to inhibit binding of a ligand of RAGE to RAGE so as to thereby reduce insulin levels in the subject.

This invention further provides a method for reducing levels of triglycerides in a subject which comprises administering to the subject an antagonist of a receptor for advanced glycation end products (RAGE) in an amount effective to inhibit binding of a ligand of RAGE to RAGE so as to thereby reduce triglyceride levels in the subject.

This invention further provides a method for reducing levels of leptins in a subject which comprises administering to the subject an antagonist of a receptor for advanced glycation end products (RAGE) in an amount effective to inhibit binding of a ligand of RAGE to RAGE so as to thereby reduce leptin levels in the subject.

### TERMS

As used herein “RAGE” means a receptor for advanced glycation end products; “sRAGE” means a soluble form of a receptor for an advanced' glycation end products, such as the extracellular two-thirds of the RAGE polypeptide, specifically the V and C domains.

As used herein “antagonist” means a compound that prevents a substantial biological response or inhibits such biological response. For example, an antagonist may prevent binding of an agonist to RAGE by occupying the same binding site or by binding to another site on the receptor so that the interaction between the RAGE agonist and RAGE is prevented. The antagonist may also prevent a biological response by acting as a non-functional decoy protein such that the RAGE agonist binds the decoy RAGE receptor rather than the functional RAGE receptor thereby preventing signal transduction through the RAGE receptor.

As used herein “agonist” means a compound that binds to a receptor to form a complex that elicits a biological response specific to the receptor bound.

“Administering” a compound can be effected or performed using any of the various methods and delivery systems known to those skilled in the art. The administering can be performed, for example, intravenously, orally, nasally, via the cerebrospinal fluid, via implant, transmucosally, transdermally, intramuscularly, intraocularly, topically and subcutaneously. The following delivery systems, which employ a number of routinely used pharmaceutically acceptable carriers, are only representative of the many embodiments envisioned for administering compositions according to the instant methods.

Injectable drug delivery systems include solutions, suspensions, gels, microspheres and polymeric injectables, and can comprise excipients such as solubility-altering compounds (e.g., ethanol, propylene glycol and sucrose) and polymers (e.g., polycaprylactones and PLGA's). Implantable systems include rods and discs, and can contain excipients such as PLGA and polycaprylactone.

Oral delivery systems include tablets and capsules. These can contain excipients such as binders (e.g., hydroxypropylmethylcellulose, polyvinyl pyrilodone, other cellulosic materials and starch), diluents (e.g., lactose and other sugars, starch, dicalcium phosphate and cellulosic materials), disintegrating compounds (e.g., starch polymers and cellulosic materials) and lubricating compounds (e.g., stearates and talc).

Transmucosal delivery systems include patches, tablets, suppositories, pessaries, gels and creams, and can contain excipients such as solubilizers and enhancers (e.g., propylene glycol, bile salts and amino acids), and other vehicles (e.g., polyethylene glycol, fatty acid esters and derivatives, and hydrophilic polymers such as hydroxypropylmethylcellulose and hyaluronic acid).

Dermal delivery systems include, for example, aqueous and nonaqueous gels, creams, multiple emulsions, microemulsions, liposomes, ointments, aqueous and nonaqueous solutions, lotions, aerosols, hydrocarbon bases and powders, and can contain excipients such as solubilizers, permeation enhancers (e.g., fatty acids, fatty acid esters, fatty alcohols and amino acids), and hydrophilic polymers (e.g., polycarbophil and polyvinylpyrolidone). In one embodiment, the pharmaceutically acceptable carrier is a liposome or a transdermal enhancer.

Solutions, suspensions and powders for reconstitutable delivery systems include vehicles such as suspending compounds (e.g., gums, zanthans, cellulosics and sugars), humectants sorbitol), solubilizers (e.g., ethanol, water, PEG and propylene glycol), surfactants (e.g., sodium lauryl sulfate, Spans, Tweens, and cetyl pyridine), preservatives and antioxidants (e.g., parabens, vitamins E and C, and ascorbic acid), anti-caking compounds, coating compounds, and chelating compounds (e.g., EDTA).

In the practice of the method, administration may comprise daily, weekly, monthly or hourly administration, the precise frequency being subject to various variables such as age and condition of the subject, amount to be administered, half-life of the compound in the subject, area of the subject to which administration is desired and the like.

“Compound” shall mean any chemical entity, including, without limitation, a glycomer, a polypeptide, a fusion protein, a peptidomimetic, a carbohydrate, a lipid, an antibody, a lectin, a nucleic acid, a small molecule, and any combination thereof. “Subject” shall mean any organism including, without limitation, a mammal such as a mouse; a rat, a dog, a guinea pig, a ferret, a rabbit and a primate. In the preferred embodiment, the subject is a human being.

“Therapeutically effective amount” of a compound means an amount of the compound sufficient to treat a subject afflicted with a disorder or a complication associated with a disorder. The therapeutically effective amount will vary with the subject being treated, the condition to be treated, the compound delivered and the route of delivery. A person of ordinary skill in the art can perform routine titration experiments to determine such an amount. Depending upon the compound delivered, the therapeutically effective amount of compound can be delivered continuously, such as by continuous pump, or at periodic intervals (for example, on one or more separate occasions). Desired time intervals of multiple amounts of a particular compound can be determined without undue experimentation by one skilled in the art.

“Pharmaceutically acceptable carriers” are well known to those skilled in the art and include, but are not limited to, 0.01-0.1M and preferably 0.05M phosphate buffer, phosphate-buffered saline (PBS), or 0.9% saline. Additionally, such pharmaceutically acceptable carriers may include, but are not limited to, aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, saline and buffered media. Parenteral vehicles include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Solid compositions may comprise nontoxic solid carriers such as, for example, glucose, sucrose, mannitol, sorbitol, lactose, starch, magnesium stearate, cellulose or cellulose derivatives, sodium carbonate and magnesium carbonate. For administration in an aerosol, such as for pulmonary and/or intranasal delivery, an agent or composition is preferably formulated with a nontoxic surfactant, for example, esters or partial esters of C6 to C22 fatty acids or natural glycerides, and a propellant. Additional carriers such as lecithin may be included to facilitate intranasal delivery. Preservatives and other additives, such as, for example, antimicrobials, antioxidants, chelating agents, inert gases, and the like may also be included with all the above carriers.

“Treating” a disorder shall mean slowing, stopping or reversing the disorder's progression. In the preferred embodiment, treating a disorder means reversing the disorder's progression, ideally to the point of eliminating the disorder itself.

“Peptide,” “polypeptide” and “protein” are used interchangeably herein to describe protein molecules that may comprise either partial or full-length sequences of amino acid residues.

The term “fusion protein” refers to a protein or polypeptide that has an amino acid sequence derived from two or more proteins. The fusion protein may also include linking regions of amino acids between amino acid portions derived from separate proteins.

As used herein, a “non-RAGE polypeptide” is any polypeptide that is not derived from RAGE or a fragment thereof. Such non-RAGE polypeptides include immunoglobulin peptides, dimerizing polypeptides, stabilizing polypeptides, amphiphilic peptides, or polypeptides comprising amino acid sequences that provide “tags” for targeting or purification of the protein.

As used herein, “immunoglobulin peptides” may comprise an immunoglobulin heavy chain or a portion thereof. In one embodiment, the portion of the heavy chain may be the Fc fragment or a portion thereof. As used herein, the Fc fragment comprises the heavy chain hinge polypeptide, and the C_{H}2 and C_{H}3 domains of the heavy chain of an immunoglobulin, in either monomeric or dimeric form. Or, the C_{H}1 and Fc fragment may be used as the immunoglobulin polypeptide. The heavy chain (or portion thereof) may be derived from any one of the known heavy chain isotypes: IgG (γ), IgM (μ), IgD (δ), IgE (ε), or IgA (α). In addition, the heavy chain (or portion thereof) may be derived from any one of the known heavy chain subtypes: IgG1 (γ 1), IgG2 (γ 2), IgG3 (γ 3), IgG4 (γ 4), IgA1 (α1), IgA2 (α2), or mutations of these isotypes or subtypes that alter the biological activity. An example of biological activity that may be altered includes reduction of an isotype's ability to bind to some Fc receptors as for example, by modification of the hinge region.

The terms “identity” or “percent identical” refers to sequence identity between two amino acid sequences or between two nucleic acid sequences. Percent identity can be determined by aligning two sequences and refers to the number of identical residues (i.e., amino acid or nucleotide) at positions shared by the compared sequences. Sequence alignment and comparison may be conducted using the algorithms standard in the art (e.g. Smith and Waterman, 1981; Needleman and Wunsch, 1970; Pearson and Lipman, 1988) or by computerized versions of these algorithms (Wisconsin Genetics Software Package Release 7.0, Genetics Computer Group, 575 Science Drive, Madison, Wis.) publicly available as BLAST and FASTA. Also, ENTREZ, available through the National Institutes of Health, Bethesda Md., may be used for sequence comparison. In one embodiment, the percent identity of two sequences may be determined using GCG with a gap weight of 1, such that each amino acid gap is weighted as if it were a single amino acid mismatch between the two sequences.

As used herein, the term “conserved residues” refers to amino acids that are the same among a plurality of proteins having the same structure and/or function. A region of conserved residues may be important for protein structure or function. Thus, contiguous conserved residues as identified in a three-dimensional protein may be important for protein structure or function. To find conserved residues, or conserved regions of 3-D structure, a comparison of sequences for the same or similar proteins from different species, or of individuals of the same species, may be made.

As used herein, a polypeptide or protein “domain” comprises a region along a polypeptide or protein that comprises an independent unit. Domains may be defined in terms of structure, sequence and/or biological activity. In one embodiment, a polypeptide domain may comprise a region of a protein that folds in a manner that is substantially independent from the rest of the protein. Domains may be identified using domain databases such as, but not limited to PFAM, PRODOM, PROSITE, BLOCKS, PRINTS, SBASE, ISREC PROFILES, SAMRT, and PROCLASS.

As used herein, “immunoglobulin domain” is a sequence of amino acids that is structurally homologous, or identical to, a domain of an immunoglobulin. The length of the sequence of amino acids of an immunoglobulin domain may be any length. In one embodiment, an immunoglobulin domain may be less than 250 amino acids. In an example embodiment, an immunoglobulin domain may be about 80-150 amino acids in length. For example, the variable region, and the C_{H}1, C_{H}2, and C_{H}3 regions of an IgG are each immunoglobulin domains. In another example, the variable, the C_{H}1, C_{H}2, C_{H}3 and C_{H}4 regions of an IgM are each immunoglobulin domains.

As used herein, a “RAGE immunoglobulin domain” is a sequence of amino acids from RAGE protein that is structurally homologous, or identical to, a domain of an immunoglobulin. For example, a RAGE immunoglobulin domain may comprise the RAGE V-domain, the RAGE Ig-like C2-type 1 domain (“C1 domain”), or the RAGE Ig-like C2-type 2 domain (“C2 domain”).

As used herein, “ligand binding domain” refers to a domain of a protein responsible for binding a ligand. The term ligand binding domain includes homologues of a ligand binding domain or portions thereof. In this regard, deliberate amino acid substitutions may be made in the ligand binding site on the basis of similarity in polarity, charge, solubility, hydrophobicity, or hydrophilicity of the residues, as long as the binding specificity of the ligand binding domain is retained.

As used herein, a “ligand binding site” comprises residues in a protein that directly interact with a ligand, or residues involved in positioning the ligand in close proximity to those residues that directly interact with the ligand. The interaction of residues in the ligand binding site may be defined by the spatial proximity of the residues to a ligand in the model or structure. The term ligand binding site includes homologues of a ligand binding site, or portions thereof. In this regard, deliberate amino acid substitutions may be made in the ligand binding site on the basis of similarity in polarity, charge, solubility, hydrophobicity, or hydrophilicity of the residues, as long as the binding specificity of the ligand binding site is retained. A ligand binding site may exist in one or more ligand binding domains of a protein or polypeptide.

As used herein, a “ligand” refers to a molecule or compound or entity that interacts with a ligand binding site, including substrates or analogues or parts thereof. As described herein, the term “ligand” may refer to compounds that bind to the protein of interest. A ligand may be an agonist, an antagonist, or a modulator. Or, a ligand may not have a biological effect. Or, a ligand may block the binding of other ligands thereby inhibiting a biological effect. Ligands may include, but are not limited to, small molecule inhibitors. These small molecules may include peptides, peptidomimetics, organic compounds and the like. Ligands may also include polypeptides and/or proteins.

“Amino acid residue” means an individual monomer unit of a polypeptide chain, which result from at least two amino acids combining to form a peptide bond.

“Amino acid” means an organic acid that contains both an amine group and a carboxyl group.

The abbreviations used herein for amino acids are those abbreviations which are conventionally used: A=Ala=Alanine; R=Arg=Arginine; N=Asn=Asparagine; D=Asp=Aspartic acid; C=Cys=Cysteine; Q=Gln=Glutamine; E=Glu=Gutamic acid; G=Gly=Glycine; H=His=Histidine; I=Ile=Isoleucine; L=Leu=Leucine; K=Lys=Lysine; M=Met=Methionine; F=Phe=Phenyalanine; P=Pro=Proline; S=Ser=Serine; T=Thr=Threonine; W=Trp=Tryptophan; Y=Tyr=Tyrosine; V=Val=Valine. The amino acids may be L- or D-amino acids. An amino acid may be replaced by a synthetic amino acid which is altered so as to increase the half-life of the peptide or to increase the potency of the peptide, or to increase the bioavailability of the peptide.

The polypeptide of the present invention may comprise alterations to the sequence of human RAGE. The peptide of the present invention may comprise alterations in sequence which do not affect the functionality of the peptide in a negative way, but which may increase the functionality of the peptide in a positive way, e.g. increase the potency of the peptide. Some examples of such alterations of the first 30 amino acids (1-30) of the V-domain of human sRAGE (SEQ ID NO: 7) are listed herein below as examples:
(a) Substitute D-alanine for L-alanine in position 6;
(b) Substitute D-lysine for L-lysine in position 15;
(c) Substitute D-alanine for L-alanine in position 6 and D-lysine for L-lysine in position 15;
(d) Omit amino acids 1-5 of the V domain, making the N-amino end group L-alanine;
(e) Omit amino acids 1-5 of the V domain making the N-amino acid D-alanine;
(f) Substitute D-lysine for the L-lysine in the amino acid number “30” position of the V domain of Sequence I.D. No. 5;
(g) Substitute L-arginine for L-lysine in the 30 position of the V domain;
(h) Substitute L-arginine for L-lysine in the 30 position of the V domain and add glycine as the carboxyl terminal group to produce a 31 amino acid peptide;
(i) Substitute L-arginine for L-lysine in the 30 position of the amino acid peptide containing the amino acid sequence of 6-30 described for the V domain of sRAGE;
(j) Substitute L-arginine for L-lysine in the 30 position of the amino acid peptide containing the amino acid sequence of 6-30 described for the V domain of sRAGE and add glycine as the carboxyl terminal group to produce a 25 amino acid sequence peptide;
(k) Substitute D-lysine for L-lysine in the 30 position of the 6-30 amino acid sequence designated for the V domain;
(l) Substitute D-lysine for L-lysine in the 30 position of the 6-30 amino acid sequence designated for the V domain and add L-alanine at the C-terminal position of the new 26 amino acid peptide;
(m) Substitute D-valine for L-valine in the 13 position of the V domain 30 amino acid peptide designated 6-30 of the sRAGE V domain;
(n) Substitute D-valine for L-valine in the 13 position of the 25 amino acid peptide designated 6-30 of the sRAGE V domain;
(o) Substitute D-alanine for L-alanine in the 6 position of the 30 amino acid peptide and D-valine for L-valine in the 13 position of the 30 amino acid of the V domain;
(p) Substitute D-alanine for L-alanine in the 6 position and D-valine for L-valine in the 13 position of the 25 amino acid peptide designated 6-30 of the V domain of sRAGE;
(q) the above-listed (a)-(p) peptides derivatized through the carboxylic acid of position 30 with albumin, globulins or different length peptides composed of amino acids contained within positions 31 through 281 of the human, mouse, rat or bovine sRAGE protein.

In addition to naturally-occurring forms of polypeptides derived from sRAGE, the present invention also embraces other polypeptides such as polypeptide analogs of sRAGE which have the equivalent functionality or a compound more potent or more positive functionality. Such analogs include fragments of sRAGE. Following the procedures of the published application by Alton et al. (WO 83/04053), one can readily design and manufacture genes coding for microbial expression of polypeptides having primary conformations which differ from that herein specified for in terms of the identity or location of one or more residues (e.g., substitutions, terminal and intermediate additions and deletions). Alternately, modifications of cDNA and genomic genes can be readily accomplished by well-known site-directed mutagenesis techniques and employed to generate analogs and derivatives of sRAGE polypeptide. Such products share at least one of the biological properties of sRAGE but may differ in others. As examples, products of the invention include those which are foreshortened by e.g., deletions; or those which are more stable to hydrolysis (and, therefore, may have more pronounced or longer lasting effects than naturally-occurring); or which have been altered to delete or to add one or more potential sites for O-glycosylation and/or N-glycosylation or which have one or more cysteine residues deleted or replaced by e.g., alanine or serine residues and are potentially more easily isolated in active form from microbial systems; or which have one or more tyrosine residues replaced by phenylalanine and bind more or less readily to target proteins or to receptors on target cells. Also comprehended are polypeptide fragments duplicating only a part of the continuous amino acid sequence or secondary conformations within sRAGE, which fragments may possess one property of sRAGE and not others. It is noteworthy that activity is not necessary for any one or more of the polypeptides of the invention to have therapeutic utility or utility in other contexts, such as in assays of sRAGE antagonism. Competitive antagonists may be quite useful in, for example, cases of overproduction of sRAGE.

The polypeptide of the present invention may be a peptidomimetic which may be at least partially unnatural. The peptidomimetic may be a small molecule mimic of a portion of the amino acid sequence of sRAGE. The compound may have increased stability, efficacy, potency and bioavailability by virtue of the mimic. Further, the compound may have decreased toxicity. The peptidomimetic may have enhanced mucosal intestinal permeability. The compound may be synthetically prepared. The of the present invention may include L-, D-, DL- or unnatural amino acids, alpha, alpha-disubstituted amino acids, N-alkyl amino acids, lactic acid (an isoelectronic analog of alanine). The peptide backbone of the compound may have at least one bond replaced with PSI-[CH═CH] (Kempf et al. 1991). The compound may further include trifluorotyrosine, p-Cl-phenylalanine, p-Br-phenylalanine, poly-L-propargylglycine, poly-D,L-allyl glycine, or poly-L-allyl glycine.

The compound may be conjugated to a carrier. The peptide or compound may be linked to an antibody, such as a Fab or a Fc fragment for specifically targeted delivery. The carrier may be a diluent, an aerosol, a topical carrier, an aqueous solution, a nonaqueous solution or a solid carrier.

When administered, compounds (such as a peptide comprising the V-domain of sRAGE) are often cleared rapidly from the circulation and may therefore elicit relatively short-lived pharmacological activity. Consequently, frequent injections of relatively large doses of bioactive compounds may by required to sustain therapeutic efficacy. Compounds modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline are known to exhibit substantially longer half-lives in blood following intravenous injection than do the corresponding unmodified compounds (Abuchowski et al., 1981; Newmark et al., 1982; and Katre et al., 1987). Such modifications may also increase the compound's solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound adducts less frequently or in lower doses than with the unmodified compound.

A RAGE protein or polypeptide may comprise full-length human RAGE protein (SEQ ID NO: 1), or a fragment of human RAGE. As used herein, a fragment of a RAGE polypeptide is at least 5 amino acids in length, may be greater than 30 amino acids in length, but is less than the full amino acid sequence. In alternate embodiments, the RAGE polypeptide may comprise a sequence that is 70%, or 80%, or 85%, or 90% identical to human RAGE, or a fragment thereof. For example, in one embodiment, the RAGE polypeptide may comprise human RAGE, or a fragment thereof, with Glycine as the first residue rather than a Methionine (see e.g., Neeper et al., 1992). Or, the human RAGE may comprise full-length RAGE with the signal sequence removed (SEQ ID NO: 2) or a portion of that amino acid sequence.

The fusion proteins of the present invention may also comprise sRAGE (SEQ ID NO: 3), a polypeptide 90% identical to sRAGE, or a fragment of sRAGE. As used herein, sRAGE is the RAGE protein that does not include the transmembrane region or the cytoplasmic tail (Park et al., 1998). For example, the RAGE polypeptide may comprise human sRAGE, or a fragment thereof, with Glycine as the first residue rather than a Methionine (see e.g., Neeper et al., 1992). Or, a RAGE polypeptide may comprise human sRAGE with the signal sequence removed (SEQ ID NO: 4) or a portion of that amino acid sequence.

The following are examples of forms of soluble RAGE: mature human soluble RAGE, mature bovine soluble RAGE, and mature murine soluble RAGE. Representative portions of sRAGE include, but are not limited to, peptides having an amino acid sequence which corresponds to amino acid numbers (2-30), (5-35), (10-40), (15-45), (20-50), (25-55), (30-60), (30-65), (10-60), (8-100), 14-75), (24-80), (33-75), (45-110) of human sRAGE protein. The 22 amino acid leader sequence of immature human RAGE is Met Ala Ala Gly Thr Ala Val Gly, Ala Trp Val Leu Val Leu Ser Leu Trp Gly Ala Val Val Gly (SEQ ID NO: 12).

For example, embodiments of the present invention provide fusion proteins comprising a RAGE polypeptide linked to a second, non-RAGE polypeptide. In one embodiment, the fusion protein may comprise a RAGE ligand binding site. In an embodiment, the ligand binding site comprises the most N-terminal domain of the fusion protein. The RAGE ligand binding site may comprise the V domain of RAGE, or a portion thereof. In an embodiment, the RAGE ligand binding site comprises SEQ ID NO: 6 or a sequence 90% identical thereto, or SEQ ID NO: 8 or a sequence 90% identical thereto.

In an embodiment, the RAGE polypeptide may be linked to a polypeptide comprising an immunoglobulin domain or a portion (e.g., a fragment thereof) of an immunoglobulin domain. In one embodiment, the polypeptide comprising an immunoglobulin domain comprises at least a portion of at least one of the C_{H}2 or the C_{H}3 domains of a human IgG.

In other embodiments, the RAGE protein may comprise a RAGE V domain (SEQ ID NO: 5) (Neeper et al., 1992; Schmidt et al., 1997). Or, a sequence 90% identical to the RAGE V domain or a fragment thereof may be used.

Or, the RAGE protein may comprise a fragment of the RAGE V domain. In one embodiment the RAGE protein may comprise a ligand binding site. In an embodiment, the ligand binding site may comprise SEQ ID NO: 6, or a sequence 90% identical thereto, or SEQ ID NO: 8, or a sequence 90% identical thereto. In yet another embodiment, the RAGE fragment is a synthetic peptide.

Thus, the RAGE polypeptide used in the fusion proteins of the present invention may comprise a fragment of, full length RAGE. As is known in the art, RAGE comprises three immunoglobulin-like polypeptide domains, the V domain, and the C1 and C2 domains each linked to each other by an interdomain linker. Full-length; RAGE also includes a transmembrane polypeptide and a cytoplasmic tail downstream (C-terminal) of the C2 domain, and linked to the C2 domain.

Examples of fusion proteins include polypeptides comprising (i) the V-domain of sRAGE linked to the CH2 and CH3 domains (i.e. Fc domain) of an Ig, and (ii) the V-domain and C1 domain of sRAGE linked to the CH2 and CH3 domains of an Ig. In these two examples, the fusion of part (i) can comprise, for example, about 250 amino acid residues (with about 136 residues belonging to the sRAGE V-domain), and the fusion protein of part (ii) can comprise, for example, about 380 amino acid residues. In one embodiment of each of the fusion proteins of parts (i) and (ii), the sRAGE V-domain-containing portion of the fusion protein comprises an amino acid sequence (e.g. about 30 amino acid residues) which permits, binding to Aβ peptide. Such sequence can be, for example, A-Q-N-I-T-A-R-I-G-E-P-C-V-L-K-C-K-G-A-P-K-K-P-P-Q-R-L-E-W-K (SEQ ID NO: 6) (see, e.g. U.S. Pat. No. 6,555,651 and U.S. patent application Ser. No. 11/197,644), or the first ten residues thereof. This invention will be better understood from the Experimental Details which follow. However, one skilled in the art will readily appreciate that the specific methods and results discussed are merely illustrative of the invention as described more fully in the claims which follow thereafter.

### EXPERIMENTAL DETAILS

### Materials and Methods

Wild-type C57BL/6 mice or RAGE null CB7BL/6 mice were used in these experiments. Animals were fed high fat diet (60W fat) or control chow regular diets (11.8% fat) prepared by Research Diets. Wild-type C57BL/6 mice were purchased from the Jackson labs or bred in house. The RAGE null mice were backcrossed more than twelve generations into C57BL/6 and were bred in house.

Murine sRAGE was prepared in a baculovirus expression system using Sf9 cells, purified to homogeneity, devoid of endotoxin, and sterile-filtered (0.2 μm) according to procedures published previously (Park et al., 1998)

### Results

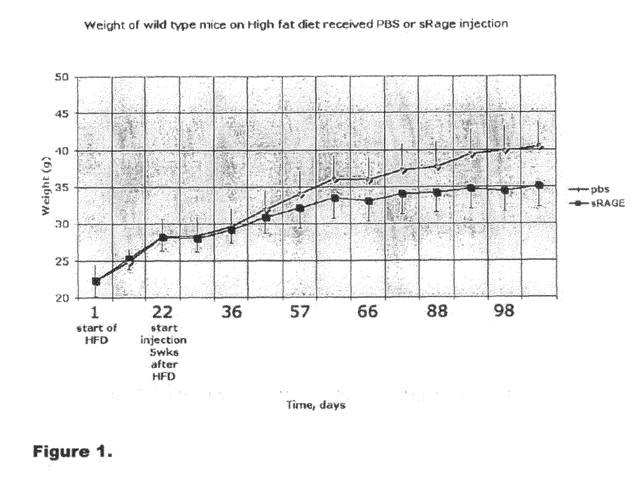

Wild-type C57BL/6 mice were started on a high fat diet on day 1 of the experiment. On day 31, the animals were treated with either soluble RAGE, 150 μg every other day by intraperitoneal route, or by vehicle, phosphate buffered saline (equal volumes per day). The weights of the animals were recorded as shown in FIG. 1. The sRAGE-treated animals (squares) displayed significantly lower weights than the vehicle (PBS)-treated animals (diamonds; p<0.05 sRAGE-versus vehicle (PBS)-treated animals). All animals continued to receive and consume the high fat diet.

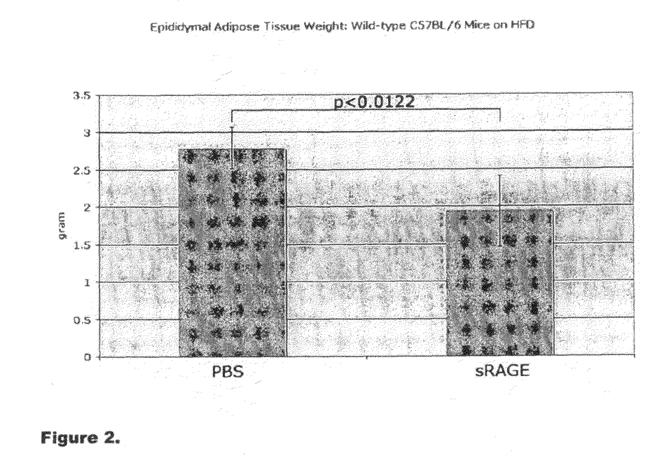

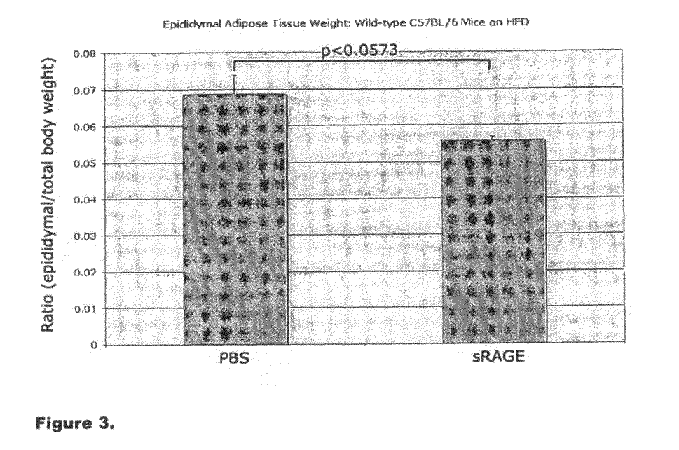

When epididymal adipose tissue was retrieved from these mice, the adipose tissue weight (FIG. 2) and the adipose tissue weight to body weight ratio (FIG. 3) was significantly lower in the sRAGE-treated mice versus the vehicle (PBS)-treated mice (p<0.0573).

To determine if RAGE, itself, was the key factor mediating the weight gain response to high fat diet feeding, homozygous RAGE null mice and RAGE-expressing mice, all in the C57BL/6 background, were used in the following studies.

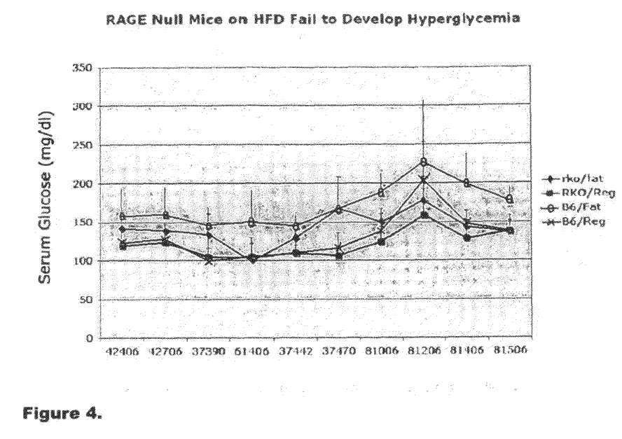

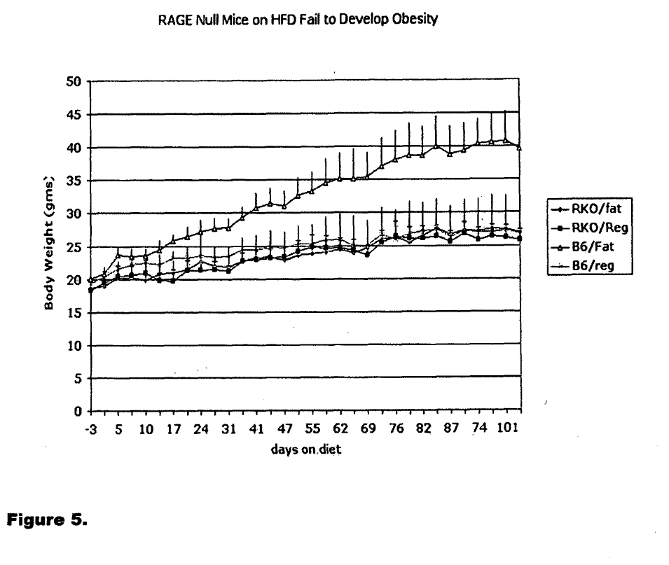

Wild-type C57BL/6 mice and RAGE null mice in the C57BL/6 background (indicated “RKO” in the figures) were fed regular chow (“reg”) or high fat diet (“fat”). The mice were followed serially. As illustrated in FIG. 4, although wild-type C57BL/6 mice fed high fat diet (triangles) developed frank hyperglycemia and diabetes over the time course, RAGE null mice fed and consuming the same high fat diet failed to develop hyperglycemia over the same time course (diamonds); p<0.05. The RAGE null mice and C57BL/6 mice fed regular chow (X's and squares, respectively) displayed identical glucose levels on this time course; these levels were not different from RAGE null mice fed high fat diet (p>0.05). In parallel with these observations, body weights in the RAGE null mice fed high fat diet (FIG. 5, diamonds) were significantly lower than the wild-type C57BL/6 mice fed the high fat diet (FIG. 4, triangles; p<0.05). Notably, body weight in RAGE null mice fed high fat diet was not significantly different from C57BL/6 mice fed regular chow (FIG. 5, X's) of RAGE null mice fed regular chow (FIG. 5, squares); p>0.05.

Male, six week old, RAGE null mice (indicated RAGE 0 in Table 1) or wild-type C57BL/6 (indicated WT in Table 1) were assigned either regular chow (Low Fat, 11.8% kcal) or high-fat chow (High Fat, 60% kcal) and followed for sixteen weeks. At sacrifice, there were no significant differences in metabolic or physical characteristics between regular chow fed wild-type C57BL/6 mice versus regular chow fed RAGE null mice (Table 1). On high-fat chow, wild-type mice displayed significantly increased fasting glucose, leptin, leptin/percent body fat, and cholesterol levels as compared to RAGE null mice on high-fat chow. Rage null mice displayed significantly lower body mass, lean mass, and percent body fat on high-fat chow as compared to wild-type C57/BL6 mice on high-fat chow (Table 1). There was no difference in food consumption or kcal/body mass between wild-type C57BL/6 and RAGE null mice on high-fat chow.

TABLE 1
RAGE null mice on either high or low fat diets have
lower insulin, triglyceride, cholesterol and leptin levels as
compared to wildtype C57BL/6 mice on either high or low fat diets.

| | | Final Body | Lean | | | | | | Leptin % |
|---|---|---|---|---|---|---|---|---|---|
| | | Mass | Mass | Fat | Insulin | Triglyceride | Cholesterol | Leptin | Body |
| Genotype | Diet | (g) | (g) | (%) | (μg/L) | (mg/dL) | (mmol/L) | (ng/mL) | Fat |
| RAGE 0 | High Fat | 26.7 ± 5.7 | 19.13 ± 2.12 | 26.88 ± 6.63 | 0.43 ± 0.14 | 22.18 ± 6 | 65.5 ± 4.97 | 1.98 ± 0.87 | 0.08 |
| (n = 8) | | | | | | | | | |
| RAGE 0 | Low | 25.8 ± 1.6 | 21.34 ± 1.8 | 14.5 ± 1.15 | 0.37 ± 0.06 | 23.89 ± 4.12 | 27.29 ± 8.05 | 0.57 ± 0.19 | 0.03 |
| (n = 7) | Fat | | | | | | | | |
| WT | High | 39.34 ± 4.6* | 24.19 ± 1.93* | 36.59 ± 6.5* | 0.69 ± 0.29 | 21.94 ± 7.21 | 102.92 ± 17.22* | 9.39 ± 5.66* | 0.25 |
| (n = 7) | Fat | | | | | | | | |
| WT (n = 8) | Low Fat | 27.15 ± 1.5 | 21.71 ± 1.4 | 16.7 ± 1.52 | 0.54 ± 0.26 | 28.16 ± 6.1 | 39.51 ± 7.77 | 1.09 ± 0.45 | 0.06 |

(*Significantly different than other three groups using Tukey-Kramer HSD Analysis).

### Discussion

The results of these studies implicate RAGE in the development of obesity and consequent hyperglycemia induced by high-fat feeding and demonstrate that blockade of RAGE with sRAGE (which prevents access of ligands to the receptor by acting as a soluble decoy) can suppress the maladaptive impact of a high-fat diet on body mass and metabolism in murine models. Consequently, administration of a compound that blocks RAGE from interacting with its ligands might present a novel form of therapeutic intervention for the treatment of obesity as well as resulting complications which emerge in obese individuals.

### REFERENCES

U.S. Pat. No. 7,087,632 issued Aug. 8, 2006; Mjalli et al.
U.S. Pat. No. 6,613,801 issued Sep. 2, 2003; Mjalli et al.
U.S. Pat. No. 6,555,651 issued Apr. 29, 2005; Stern et al.
U.S. patent application Ser. No. 11/197,644, filed Aug. 3, 2005, Mjalli et al.
U.S. patent application Ser. No. 11/511,163, filed Aug. 28, 2006; Mjalli, et al.
PCT International Application Publication No. WO/1983/004053, published Nov. 24, 1983; Alton et al.
Abuchowski, A. et al. Reduction of plasma urate levels in the cockerel with polyethylene glycol-uricase. J Pharmacol Exp Ther. 1981 November; 219(2):352-4.
Brownlee, M., et al., Advanced glycosylation endproducts in tissue and the biochemical basis of diabetic complications. N. Engl. J. Med. 1988 318:1315-1320.
De Souza, C T., et al. Consumption of a fat-rich diet activates a proinflammatory response and induces insulin resistance in the hypothalamus. Endocrinology 2005 146(10):4189-91.
Hofmann, M. et al. EN-RAGE (Extracellular Novel RAGE binding protein) activates endothelial cells and macrophages to mediate inflammatory responses. Circ. (Suppl). 98, #1657.
Hori, O., et al. The receptor for advanced glycation endproducts (RAGE) is a cellular binding site for amphoterin: mediation of neurite outgrowth and coexpression of RAGE and amphoterin in the developing nervous system. J. Biol. Chem. 1995 270:25752-25761.
Katre, N V., et al. Chemical modification of recombinant interleukin 2 by polyethylene glycol increases its potency in the murine Meth A sarcoma model. Proc Natl Acad Sci USA. 1987 March; 84(6):1487-91.
Kempf, D J., et al. Stereocontrolled synthesis of psi[CH═CH] dipeptide isosteres. Int J Pept Protein Res. 1991 September; 38(3):237-41.
Kokkola, R., et al. RAGE is the major receptor for the proinflammatory activity of HMGB1 in rodent macrophages. Scand J. Immunol. 2005 January; 61(1):1-9.
Lander, H L., et al., Activation of the Receptor for Advanced Glycation Endproducts triggers a MAP kinase pathway regulated by oxidant stress. J. Biol. Chem. 1997 272:17810-17814.
Liliensiek et al., J. Clin. Invest., 113:1641-50 (2004).
Needleman and Wunsch, 1970, J. Mol. Biol. 48:443
Neeper, M., et al. Cloning and expression of RAGE: a cell surface receptor for advanced glycosylation end products of proteins. J. Biol. Chem. 1992 267:14998-15004.
Newmark, et al. J. Appl. Biochem. 1982 4:185-189.
Park, L., et al. Suppression of accelerated diabetic atherosclerosis by soluble Receptor for AGE (sRAGE). Nature Medicine 1998 4:1025-1031.
Pearson and Lipman, 1988, Proc. Natl. Acad. Sci., USA, 85:2444.
Sell, D., and Monnier, V. Structure elucidation of a senescence cross-link from human extracellular matrix: implication of pentoses in the aging process. J. Biol. Chem. 1989 264:21597-21602.
Schafer, B W., And Heinzmann, C W. The S100 family of EF-hand calcium-binding proteins: functions and pathology. TIBS 1996 21:134-140.
Schleicher et al., J. Clin. Invest., 99 (3): 457-468 (1997).
Schmidt et al., Circ. (Suppl.) 96#194 (1997).
Schmidt et al., Nature Med., 1:1002-1004 (1995).
Schmidt, A M., et al. Isolation and characterization of binding proteins for advanced glycosylation endproducts from lung tissue which are present on the endothelial surface. J. Biol. Chem. 1992 267: 14987-14997.
Selkoe, D. Alzheimer's disease: a central role for amyloid. J. Neuropathol. Exp. Neurol. 1994 53:438-447.
Sisodia, S., and Price, D. Role of beta-amyloid protein in Alzheimer's disease. FASEB J. 1995 9:366-370.
Smith and Waterman, 1981, Adv. Appl. Math. 2:482.
Wautier, J-L., et al. Receptor-mediated endothelial cell dysfunction in diabetic vasculopathy: soluble receptor for advanced glycation endproducts blocks hyperpermeability. J. Clin. Invest. 1996 97: 238-243.
Yan, S D., et al. Enhanced cellular oxidant stress by the interaction of advanced glycation endproducts with their receptors/binding proteins. J. Biol. Chem. 1994 269:9889-9897.
Yan, S D., et al. RAGE and amyloid beta peptide neurotoxicity in Alzheimer's disease. Nature 1996 382:685-691.
Yan, S D., et al. Amyloid-beta-RAGE interaction elicits neuronal expression of M-CSF: a proinflammatory pathway in Alzheimer's disease. Proc. Natl. Acad. Sci. 1997 94:5296-5301.
Zimmer, D B., et al. The S100 protein family: history, function, and expression. Brain Research Bulletin 1995 37:417-429.

What is claimed is:

1. A method for treating obesity or hyperglycemia in a subject which comprises administering to the subject an antagonist of a receptor for advanced glycation end products (RAGE) in an amount effective to inhibit binding of a ligand of RAGE to RAGE so as to thereby treat obesity or hyperglycemia in the subject.

2. (canceled)

3. The method of claim 1, wherein the antagonist is a polypeptide.

4. The method of claim 3, wherein the polypeptide is a soluble fragment of RAGE.

5. The method of claim 4, wherein the soluble fragment of RAGE is sRAGE

6. The method of claim 5, wherein the soluble fragment of sRAGE is a V-domain of sRAGE or a fragment of the V-domain which retains the ability to inhibit the binding of a ligand of RAGE to sRAGE.

7. (canceled)

8. (canceled)

9. The method of claim 1, wherein the antagonist comprises a fusion protein comprised of a RAGE polypeptide linked to a second, non-RAGE polypeptide wherein the RAGE polypeptide comprises a RAGE ligand binding site.

10. The method of claim 9, wherein the RAGE polypeptide is linked to a polypeptide comprising an immunoglobulin domain or a portion of an immunoglobulin domain.

11. The method of claim 10, wherein the polypeptide comprising the immunoglobulin domain comprises at least a portion of at least one of the C_{H}2 or C_{H}3 domains of a human IgG.

12. The method of claim 9, wherein the RAGE ligand binding site comprises consecutive amino acids comprising the sequence A-Q-N-I-T-A-R-I-G-E-P-L-V-L-K-C-K-G-A-P-K-K-P-P-Q-R-L-E-W-K (SEQ ID NO. 6) or a sequence 90% identical thereto or Q-N-I-T-A-R-I-G-E-P-L-V-L-K-C-K-G-A-P-K-K-P-P-Q-R-L-E-W-K (SEQ ID NO. 8) or a sequence 90% identical thereto.

13. The method of claim 9, wherein the RAGE polypeptide comprises consecutive amino acids corresponding to amino acids 24-116 of human RAGE (SEQ ID NO: 9).

14. The method of claim 9, wherein the RAGE polypeptide comprises consecutive amino acids corresponding to amino acids 24-123 of human RAGE (SEQ ID NO: 10).

15. The method of claim 9, wherein the RAGE polypeptide comprises consecutive amino acids corresponding to amino acids 24-226 of human RAGE (SEQ ID NO: 11).

16. The method of claim 9, wherein the RAGE polypeptide comprises consecutive amino acids corresponding to amino acids 24-339 of human RAGE (SEQ ID NO: 4).

17. The method of claim 1, wherein the antagonist comprises a RAGE fusion protein and a pharmaceutically acceptable carrier, wherein the RAGE fusion protein comprises a RAGE polypeptide linked to a second, non-RAGE polypeptide wherein the RAGE polypeptide comprises a RAGE ligand binding site.

18.-21. (canceled)

22. The method of claim 1, wherein the antagonist is a small molecule.

23. (canceled)

24. (canceled)

25. The method of claim 1, wherein the antagonist is a compound having the structure 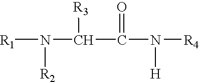
wherein
R₁ and R₂ are independently selected from
a) —H;
b) —C₁₋₆ alkyl;
c) -aryl;
d) —C₁₋₆ alkylaryl;
e) —C(O)—O—C₃₋₆ alkyl;
f) —C(O)—O—C₁₋₆ alkylaryl;
h) —C(O)—NH—C₁₋₆ alkylaryl;
i) —SO₂—C₁₋₆ alkyl;
j) —SO₂—C₁₋₆ alkylaryl;
k) —SO₂-aryl;
l) —SO₂—NH—C₁₋₆ alkyl;
m) —SO₂—NH—C₁₋₆ alkylaryl;
n) 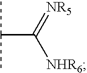
o) —C(O)—C₁₋₆ alkyl; and
p) —C(O)—C₁₋₆ alkylaryl;
R₃ is selected from
(a) -aryl; and
(b) —C₁₋₃ alkylaryl,
wherein aryl is substituted by C₁₋₆ alkyl, C₁₋₆ alkoxy, C₁₋₆ alkylaryl, or C₁₋₆ alkoxyaryl;
R₄ is selected from 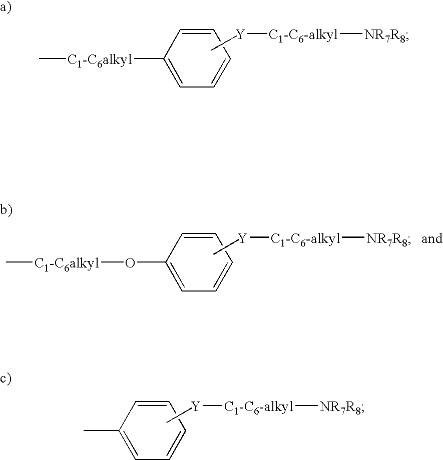
R₅ and R₆ are independently selected from the group consisting of hydrogen, C₁-C₆ alkyl, C₁-C₆ alkylaryl, and aryl; and wherein
the aryl and/or alkyl group(s) in R₁, R₂, R₄, R₅, R₆, R₇, R₈, R₉, R₁₀, R₁₈, R₁₉, and R₂₀ may be optionally substituted 1-4 times with a substituent group, wherein said substituent group(s) or the term substituted refers to groups selected from the group consisting of:
a) —H;
b) alkyl;
—Y-aryl;
—Y—C₁₋₆ alkylaryl;
—Y—C₁₋₆-alkyl-NR₇R₈; and
—Y—C₁₋₆; and
c) halogen, hydroxyl, cyano, carbamoyl, or carboxyl; and
wherein
Y and W are independently selected from the group consisting of —CH₂—N(H), SO₂—, —CON(H)—, —NHC(O)—, —NHCON(H)—, —NHSO₂—SO₂N(H)—, —C(O)—O—, —NHSO₂NH—, —O—CO—, 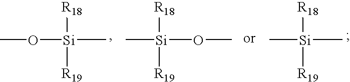
R₁₈ and R₁₉ are independently selected from the group consisting of aryl, C1-C₆ alkyl, C1-C₆ alkylaryl, C1-C₆ alkoxy, and C1-C₆ alkoxyaryl;
R₂₀ is selected from the group consisting of aryl, C1-C₆ alkyl, and C1-C₆ alkylaryl;
R₇, R₈, R₉ and R₁₀ are independently selected from the group consisting of hydrogen, aryl C1-C₆ alkyl, and C1-C₆ alkylaryl; and wherein
R.sub.7 and R.sub.8 may be taken together to form a ring having the formula —(CH₂)ₘ—X—(CH₂)ₙ-bonded to the nitrogen atom to which R₇ and R₈ are attached, and/or R₅ and R₆ may, independently, be taken together to form a ring having the formula —(CH₂)ₘ—X—(CH₂)ₙ— bonded to the nitrogen atoms to which R₅ and R₆ are attached, wherein m and n are, independently, 1, 2, 3, or 4; X is selected from the group consisting of —CH₂—, —O—, —S—, —S(O₂)—, —C(O)—, —CON(H)—, —NHC(O)—, —NHCON(H)—, —NHSO₂—, —SO₂N(H)—, —C(O)—O—, —O—C(O)—, —NHSO₂NH—, 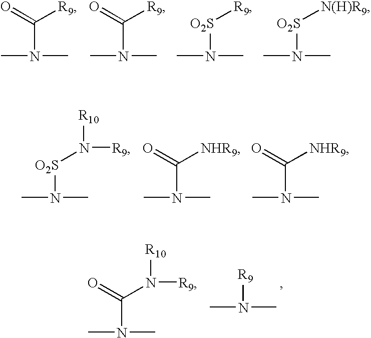
or a pharmaceutically acceptable salt thereof.

26. The method of claim 1, wherein the RAGE antagonist is 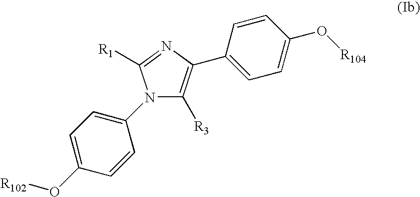
wherein
R₁ is -hydrogen, -alkyl, or -alkenyl,
R₃ is -hydrogen or -alkyl; and
R₁₀₂ and R₁₀₄ are independently selected from the group consisting of:
a) —H;
b) -alkyl;
c) -aryl;
d) -heteroaryl;
e) -alkylene-heteroarylene-aryl;
f) -alkylene-aryl;
g) -alkylene-W₂—R₁₈;
h) —Y₄—NR₂₃R₂₄;
i) —Y₄—NH—C(═NR₂₅)NR₂₃R₂₄;
j) —Y₄—C(═NR₂₅)NR₂₃R₂₄; and
k) —Y₄—Y₅-A₂;
wherein
W₂ is —CH₂—, —O—, —N(H), —S—, SO₂—, —CON(H)—, —NHC(O)—, —NHCON(H)—, —NHSO₂—, —SO₂N(H)—, —C(O)—O—, —NHSO₂NH—, —O—S(O)₂—, —O—CO—, 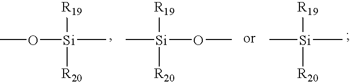
wherein R₁₉ and R₂₀ are independently selected from the group consisting of: -hydrogen, -aryl, -alkyl, -alkylene-aryl, alkoxy, and -alkylene-O-aryl;
R₁₈ is -aryl, -alkyl, -alkylene-aryl, -alkylene-heteroaryl, or -alkylene-O-aryl;
Y₅ is a direct bond, —CH₂—, —O—, —N(H), —S—, SO₂—, —C(O)—, —CON(H)—, —NHC(O)—, —NHCON(H)—, —NHSO₂—, —SO₂N(H)—, —C(O)—O—, —NHSO₂NH—, —O—CO—, 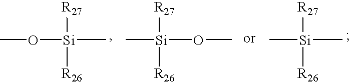
wherein R₂₇ and R₂₆ are independently selected from the group consisting of -aryl, -alkyl, -alkylene-aryl, alkoxy, and -alkyl-O-aryl;
Y₄ is
a) -alkylene;
b) -alkenylene;
c) -alkynylene;
d) -arylene;
e) -heteroarylene;
f) -cycloalkylene;
g) -heterocyclylene;
h) -alkylene-arylene;
i) -alkylene-heteroarylene;
j) -alkylene-cycloalkylene;
k) -alkylene-heterocyclylene;
l) -arylene-alkylene;
m) -heteroarylene-alkylene;
n) -cycloalkylene-alkylene;
o) -heterocyclylene-alkylene;
p) —S(O)₂—; or
g) —S(O)—;
wherein said alkylene groups may optionally contain one or more O, S, S(O), or SO₂ atoms;
A₂ is
a) heterocyclyl, fused arylheterocyclyl, or fused heteroarylheterocyclyl, containing at least one basic nitrogen atom, or
b) -imidazolyl,
R₂₃, R₂₄, and R₂₅ are independently selected from the group consisting of: -hydrogen, -aryl, -heteroaryl, -alkylene-heteroaryl, alkyl, -alkylene-aryl, -alkylene-O-aryl, and -alkylene-O-heteroaryl; and R₂₃ and R₂₄ may be taken together to form a five membered ring having the formula —(CH₂)ₛ—X₃—(CH₂)ₜ— bonded to the nitrogen atom to which R₂₃ and R₂₄ are attached
wherein
and t are, independently, 1, 2, 3, or 4;
X₃ is a direct bond, —CH₂—, —O—, —S—, —S(O)₂—, —C(O)—, —CON(H)—, —NHC(O)—, —NHCON(H)—, —NHSO₂—, —SO₂N(H)—, —C(O)—O—, —O—C(O)—, —NHSO₂NH—, 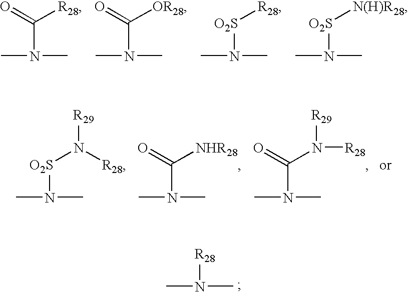
wherein R₂₈ and R₂₉ are independently selected from the group consisting of: -hydrogen, -aryl, -heteroaryl, -alkyl, -alkylene-aryl, and -alkylene-heteroaryl;
wherein
the alkyl, alkylene, alkenyl, heteroaryl, heteroarylene, cycloalkylene, heterocyclylene, arylene, fused arylheterocyclyl, fused heteroarylheterocyclyl, and/or aryl groups of R₁, R₃, R₂₃, R₂₄, R₂₅, A₂, Y₄R₁₀₂ and R₁₀₄ may be optionally substituted 1-4 times with a substituent group independently selected from the group consisting of:
a) halogen;
b) haloalkyl;
c) alkyl;
d) cyano;
e) alkyloxy;
f) aryl; and
g) aryloxy
wherein
at least one of R₁₀₂ and R₁₀₄ is a group of the formula
—Y₄—NR₂₃R₂₄,
—Y₄—NH—C(═NR₂₅)NR₂₃R₂₄,
—Y₄—C(═NR₂₅)NR₂₃R₂₄, or
—Y₄—Y₅-A₂,
with the proviso that no more than one of R₂₃, R₂₄, and R₂₅ is aryl or heteroaryl;
or a pharmaceutically acceptable salt thereof.

27.-30. (canceled)

31. The method of claim 1, wherein the RAGE antagonist is a compound selected from the group consisting of:
(20) {3-[4-(2-butyl-4-{4-[2-(4-chloro-phenyl)-ethoxy]-phenyl}-imidazol-1-yl)-phenoxy]-propyl}-diethyl-amine;
(21) {3-[4-(4-{(4-[2-(4-chloro-phenyl)-ethoxy]-phenyl}-2-isobutyl-imidazol-1-yl)-phenoxy]-propyl}-diethyl-amine;
(22) [3-(4-{2-butyl-1-[4-(4-chloro-phenoxy)-phenyl]-1H-imidazol-4-yl}-phenoxy)-propyl]-diethyl-amine;
(23) 3-(4-{2-butyl-1-[4-(4-fluoro-3-trifluoromethyl-phenoxy)-phenyl]-1H-imidazol-4-yl}-phenoxy)-propyl]-diethyl-amine;
(24) diethyl-[3-(4-{1-[4-(4-fluoro-3-trifluoromethyl-phenoxy)-phenyl]-2-methyl-1H-imidazol-4-yl}-phenoxy)-propyl]-amine;
(25) [3-(4-{2-butyl-1-[4-(3-tert-butyl-phenoxy)-phenyl]-1H-imidazol-4-yl}-phenoxy)-propyl]-diethyl-amine;
(26) (3-{4-[4-[(4-[2-(4-chloro-phenyl)-ethoxy]-phenyl}-2-(1-ethyl-propyl)-imidazol-1-yl]-phenoxy]-propyl)-diethyl-amine;
(27) {3-[4-(4-{4-[2-(4-chloro-phenyl)-ethoxy]-phenyl}-2-isobutyl-5-methyl-imidazol-1-yl)-phenoxy]-propyl}-diethyl-amine;
(28) {3-[4-(4-{4-[2-(4-chloro-phenyl)-ethoxy]-phenyl}-2-isobutyl-5-propyl-imidazol-1-yl)-phenoxy]-propyl}-diethyl-amine;
(29) {3-[4-(5-butyl-4-{4-[2-(4-chloro-phenyl)-ethoxy]-phenyl}-2-isobutyl-imidazol-1-yl)-phenoxy]-propyl}-diethyl-amine;
(30) [3-(4-{1-[4-(4-chloro-phenoxy)-phenyl]-2-isobutyl-1H-imidazol-4-yl}-phenoxy)-propyl]-diethyl-amine;
(31) [3-(4-{2-butyl-1-[4-(4-chloro-phenoxy)-phenyl]-5-methyl-1H-imidazol-4-yl}-phenoxy)-propyl]-diethyl-amine;
(32) [3-(4-[(2-butyl-1-[4-(4-chloro-phenoxy)-phenyl]-5-propyl-1H-imidazol-4-yl]-phenoxy)-propyl]-diethyl-amine;
(33) [3-(4-{2,5-dibutyl-1-[4-(4-chloro-phenoxy)-phenyl]-1H-imidazol-4-yl}-phenoxy)-propyl]-diethyl-amine;
(34) 2-butyl-1-[4-(4-chloro-phenoxy)-phenyl]-4-[4-(2-pyrrolidin-1-yl-ethoxy)-phenyl]-1H-imidazole;
(35) [3-(4-{2-butyl-4-[4-(4-chloro-phenoxy)-phenyl]-imidazol-1-yl}-phenoxy)-propyl]-dimethyl-amine;
(36) (3-{4-[2-butyl-1-(4-p-tolyloxy-phenyl)-1H-imidazol-4-yl]-phenoxy}-propyl)-diethyl-amine;
(37) [3-(4-{2-butyl-1-[4-(4-fluoro-phenoxy)-phenyl]-1H-imidazol-4-yl}-phenoxy)-propyl]-diethyl-amine;
(38) [3-(4-{4-[4-(3,3-diphenyl-propoxy)-phenyl]-2-isobutyl-imidazol-1-yl}-phenoxy)-propyl]-diethyl-amine, and
pharmaceutically acceptable salts thereof.

32. (canceled)

33. A method for reducing levels of cholesterol, insulin, triglycerides or leptins in a subject which comprises administering to the subject an antagonist of a receptor for advanced glycation end products (RAGE) in an amount effective to inhibit binding of a ligand of RAGE to RAGE so as to thereby reduce cholesterol, insulin, triglyceride or leptin levels in the subject.

34.-36. (canceled)


SEQUENCE LISTING (12 sequences)

SEQ ID NO: 1 - protein, 404 aa, Human
MAAGTAVGAWVLVLSLWGAVVGAQNITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQPRVWEPVPLEEVQLVVEPEGGAVAPGGTVTLTCEVPAQPSPQIHWMKDGVPLPLPPSPVLILPEIGPQDQGTYSCVATHSSHGPQESRAVSISIIEPGEEGPTAGSVGGSGLGTLALALGILGGLGTAALLIGVILWQRRQRRGEERKAPENQEEEEERAELNQSEEPEAGESSTGGP

SEQ ID NO: 2 - protein, 382 aa, Human
AQNITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQPRVWEPVPLEEVQLVVEPEGGAVAPGGTVTLTCEVPAQPSPQIHWMKDGVPLPLPPSPVLILPEIGPQDQGTYSCVATHSSHGPQESRAVSISIIEPGEEGPTAGSVGGSGLGTLALALGILGGLGTAALLIGVILWQRRQRRGEERKAPENQEEEEERAELNQSEEPEAGESSTGGP

SEQ ID NO: 3 - protein, 339 aa, Human
MAAGTAVGAWVLVLSLWGAVVGAQNITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQPRVWEPVPLEEVQLVVEPEGGAVAPGGTVTLTCEVPAQPSPQIHWMKDGVPLPLPPSPVLILPEIGPQDQGTYSCVATHSSHGPQESRAVSISIIEPGEEGPTAGSVGGSGLG

SEQ ID NO: 4 - protein, 317 aa, Human
AQNITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQPRVWEPVPLEEVQLVVEPEGGAVAPGGTVTLTCEVPAQPSPQIHWMKDGVPLPLPPSPVLILPEIGPQDQGTYSCVATHSSHGPQESRAVSISIIEPGEEGPTAGSVGGSGLG

SEQ ID NO: 5 - protein, 94 aa, Human
AQNITARIGEPLVLKCKGAPKKPPQRLEWKLNTGRTEAWKVLSPQGGGPWDSVARVLPNGSLFLPAVGIQDEGIFRCQAMNRNGKETKSNYRVR

SEQ ID NO: 6 - protein, 30 aa, Human
AQNITARIGEPLVLKCKGAPKKPPQRLEWK

SEQ ID NO: 7 - protein, 10 aa, Human
AQNITARIGE

SEQ ID NO: 8 - protein, 29 aa, Human
QNITARIGEPLVLKCKGAPKKPPQRLEWK

SEQ ID NO: 9 - protein, 93 aa, Human
QNITARIGEPLVLKCKGAPKKPPQRLEWKLNTGRTEAWKVLSPQGGGPWDSVARVLPNGSLFLPAVGIQDEGIFRCQAMNRNGKETKSNYRVR

SEQ ID NO: 10 - protein, 100 aa, Human
QNITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGK

SEQ ID NO: 11 - protein, 203 aa, Human
QNITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQ

SEQ ID NO: 12 - protein, 21 aa, Human
MAAGTAVGWVLVLSLWGAVVG